(19) United States
(12) Patent Application Publication
(10) Pub. No.: US 2021/0196720 A1
(43) Pub. Date: Jul. 1, 2021

(54) USE OF VIBEGRON TO TREAT PAIN ASSOCIATED WITH IRRITABLE BOWEL SYNDROME

(71) Applicant: UROVANT SCIENCES GMBH, Basel (CH)

(72) Inventors: Paul N. MUDD, JR., Cary, NC (US); Cornelia HAAG-MOLKENTELLER, Irvine, CA (US); Jihao ZHOU, Rancho Santa Margarita, CA (US); Chris SCHAUMBURG, Irvine, CA (US); Jean Paul Abrian NICANDRO, Irvine, CA (US)

(73) Assignee: UROVANT SCIENCES GMBH, Basel (CH)

(21) Appl. No.: 17/057,554

(22) PCT Filed: May 23, 2019
(86) PCT No.: PCT/IB2019/054304

§ 371 Date: Nov. 20, 2020

Related U.S. Application Data
(60) Provisional application No. 62/775,751, filed on Dec. 5, 2018.
(60) Provisional application No. 62/675,747, filed on May 23, 2018.

(51) Int. Cl. A61K 31/519; A61K 9/00; A61P 1/00; A61K 45/06

(52) U.S. Cl. CPC A61K 31/519; A61K 45/06; A61P 1/00; A61K 9/0053

(57) ABSTRACT

The present disclosure is directed to a method of treating or preventing pain associated with irritable bowel syndrome comprising orally administering to a subject in need thereof vibegron.

### BACKGROUND

Irritable bowel syndrome (IBS) is characterized by recurrent abdominal pain associated with two or more of the following: defecation, a change in frequency of stool, or a change in form or appearance of stool. Approximately 80% of patients identify pain as a symptom contributing to the severity of their IBS. Pain associated with IBS, from a pathophysiologic perspective, has been linked with secretomotor nerve activity.

While there are currently approved therapies for IBS with constipation and IBS with diarrhea, these drugs do not adequately or specifically address the pain associated with IBS, and there are no approved drugs indicated specifically for IBS-associated pain. Based on the prevalence of approximately 30 million to 40 million Americans with IBS symptoms, the 80% of patients who identify pain as a symptom contributing to the severity of their IBS and the 30% of individuals with IBS symptoms who consult with their physician, it is estimated that there are approximately 7.2 to 9.6 million patients with pain associated with IBS in the United States alone. Beta-3 adrenergic receptor (β₃-AR) activation is an effective way of relaxing the detrusor in normal and pathogenic states. Functional evidence in support of an important role for the β₃-AR in pain associated with IBS emanates from studies in vivo. β₃-AR agonists have demonstrated efficacy in alleviating symptoms of pain associated with IBS. But to date, no β₃-AR agonist drugs have received marketing approval for the treatment of pain associated with IBS, at least in part because of variable efficacy in different patient populations and worsening of IBS symptoms such as diarrhea, constipation, or both.

The beta-3 adrenergic receptor is expressed in the neurons and the smooth muscle of the human colon. In vitro studies have shown that activation of the beta-3 adrenergic receptor in the colon causes the release of somatostatin from adipocytes, or fat cells, which causes visceral analgesia and inhibits secretomotor nerve activity. In a preclinical study, administration of a rat-selective beta-3 agonist caused a significant, dose-dependent decrease in abdominal arching (a sign of pain) in rats administered mustard oil to cause visceral pain. This pain reduction was reversed by pre-treatment with a somatostatin receptor antagonist, confirming the role of somatostatin in the mechanism of action, while treatment with the somatostatin receptor antagonist alone did not alter pain behavior.

Despite some success demonstrated in in vitro and in animal studies, clinical trials studying the effect of beta-3 agonists on IBS symptoms have had mixed results. In healthy volunteers, the beta-3 agonist solabegron did not significantly alter gastrointestinal or colonic transit, measurements shown to be predictive of efficacy in phase IIB or phase III trials. It also had no effect on bowel function or on plasma somatostatin concentration.

A Phase IIa study of solabegron in subjects with IBS showed mixed results in terms of efficacy. While females showed a statistically significant increase in the number of pain- and discomfort-free days compared to placebo, there was no clinical benefit seen in male subjects. In addition, while positive results in terms of stool frequency and stool consistency were seen in patients with IBS mixed subtype (IBS-M) compared to placebo, there was no difference between solabegron treatment and placebo in either of these endpoints for the IBS subtype of diarrhea (IBS-D). No meaningful conclusions could be drawn for IBS constipation subtype (IBS-C). When evaluating bowel symptoms compared to placebo in change from baseline, no benefit was demonstrated for urgency, frequency, consistency, bloating, incomplete evacuation, or straining. Subjects treated with solabegron also experienced an increase in adverse events such as nasopharyngitis, headache, nausea, and gastroenteritis compared to those who were given a placebo.

Vibegron, (6S)-N-[4-[[(2S,5R)-5-[(R)-hydroxy(phenyl)methyl]pyrrolidin-2-yl]methyl]phenyl]-4-oxo-7,8-dihydro-6H-pyrrolo[1,2-a]pyrimidine-6-carboxamide, is a potent and highly selective beta-3 adrenergic receptor agonist demonstrating >9,000 fold selectivity for activation of β₃-AR over β₂-AR and β₁-AR in cell based in vitro assays. See Edmondson et al., J. Med. Chem. 59:609-623 (2016). Vibegron was also studied in a Phase 3 clinical trial for treating patients with overactive bladder (OAB). See Yoshida et al., European Urology 73:783-790 (2018).

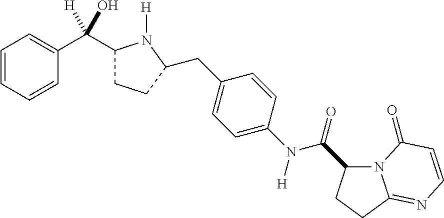

Vibegron is disclosed as a β₃-AR agonist in U.S. Pat. Nos. 8,399,480 and 8,247,415. Synthetic methods for preparing vibegron are disclosed in United States Publication Nos. US 2017/0145014, US 2015/0087832, US 2016/0176884, and US 2014/0242645. All of the cited publications are herein incorporated by reference in their entireties.

### BRIEF DESCRIPTION OF THE DRAWINGS

FIG. 1 depicts an overlay of density plots of exposure with vibegron 100 mg and 75 mg, as estimated in special populations.

FIG. 2 depicts the chemical structures of vibegron's metabolites.

### SUMMARY

The present disclosure provides a method of treating or preventing pain associated with IBS, the method comprising orally administering to a subject in need thereof vibegron.

The present disclosure further provides a pharmaceutical unit dosage composition comprising vibegron for use in a method of treating or preventing pain associated with IBS, wherein the unit dosage composition is suitable for oral administration.

### DETAILED DESCRIPTION

In order that the present disclosure can be more readily understood, certain terms are first defined. As used in this application, except as otherwise expressly provided herein, each of the following terms shall have the meaning set forth below. Additional definitions are set forth throughout the application.

In this specification and the appended claims, the singular forms “a,” “an,” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related.

The term “about” as used in connection with a numerical value throughout the specification and the claims denotes an interval of accuracy, familiar and acceptable to a person skilled in the art. Such interval of accuracy is ±10%.

The term “irritable bowel syndrome” or “IBS” as used herein means a bowel disorder typically characterized by a combination of persistent and/or recurrent abdominal pain and irregular bowel habits such as diarrhea and/or constipation. IBS is often recurrent and/or chronic and is also characterized by abnormally increased motility of the small and large intestines, producing abdominal pain, constipation, or diarrhea. One method of characterizing IBS is the Rome criteria for functional bowel disorders, including the Rome III or IV criteria. The term encompasses all classifications of irritable bowel syndrome including but not limited to each of diarrhea-predominant (IBS-D), constipation-predominant (IBS-C), mixed (IBS-M also known as alternating or IBS-A), and IBS with unknown subtype (IBS-U). IBS can include temporary or conditional bowel disorders such as that related to endometriosis and/or pregnancy. The treatment method disclosed herein can also be used for treating gastrointestinal indications such as inflammatory bowel disease, ulcerative colitis, celiac disease, and/or microscopic colitis.

Rome IV is the most recent criteria developed for diagnosis of IBS, and it increases sensitivity and specificity of the criteria with respect to abdominal pain, as compared to Rome III. See Lacy et al. “Rome Criteria and a Diagnostic Approach to Irritable Bowel Syndrome,” J. Clin. Med. 6, 99 (2017). Under Rome IV, IBS is diagnosed as: recurrent abdominal pain on average at least 1 day/week in the last 3 months, associated with two or more of the following criteria: (1) related to defecation; (2) associated with a change in the frequency of stool; and (3) associated with a change in the form (appearance) of stool. Under previously used Rome III, IBS is diagnosed as: recurrent abdominal pain or discomfort (defined as an uncomfortable sensation not described as pain) for at least 3 days/month in the last 3 months, associated with two or more of the following: (1) improvement with defecation; (2) onset associated with a change in the frequency of stool; and (3) onset associated with a change in the form (appearance) of stool. For both Rome III and IV, the criteria should be fulfilled for the last 3 months with symptoms onset at least 6 months prior to diagnosis.

The Rome criteria classify IBS into 4 distinct subtypes based on predominant stool consistency: IBS-C, IBS-D, IBS-M, and IBS-U. The definitions of diarrhea and constipation correlate with the Bristol Stool Form Scale, with Types 1 and 2 defined as constipation and Types 6 and 7 defined as diarrhea.

The term “stool consistency” as used herein means form of stool as described in the Bristol Stool Form Scale. The term encompasses all types: Type 1-Type 7 of the Bristol Stool Chart. See, e.g., Guidance for Industry: Irritable Bowel Syndrome—Clinical Evaluation of Drugs for Treatment (May 2012).

The term “impairment” as used herein means acute or chronic reduction in function. For example, renal impairment refers to a medical condition where the kidneys fail to maintain their normal function, so that waste products and metabolites accumulate in the blood.

The term “free base” as used herein refers to a basic chemical compound itself, not in the form of a salt. For example, vibegron free base refers to (65)-N-[4-[[(25,5R)-5-[(R)-hydroxy(phenyl)methyl]pyrrolidin-2-yl]methyl]phenyl]-4-oxo-7,8-dihydro-6H-pyrrolo[1,2-a]pyrimidine-6-carboxamide.

The term “pharmaceutically acceptable salt” means those salts of compounds that are safe and effective for use in subjects and that possess the desired biological activity.

Pharmaceutically acceptable salts of a basic compound can be salts of organic or inorganic acids. In some embodiments, the organic and inorganic acids include but are not limited to hydrochloric acid, hydrobromic acid, sulfuric acid, phosphoric acid, citric acid, maleic acid, mandelic acid, succinic acid and methanesulfonic acid. See generally, Journal of Pharmaceutical Science, 66, 2 (1977), which is incorporated herein by reference in its entirety.

The term “Cₘₐₓ” as used herein refers to the maximum plasma concentration of a drug after it is administered.

The term “Tₘₐₓ” as used herein refers to the time after administration of a drug when the maximum plasma concentration is reached.

The term “AUC” as used herein refers to the area under the curve of a plot of plasma concentration versus time following administration of a drug.

The term “steady state” means that the amount of the drug reaching the system is approximately the same as the amount of the drug leaving the system. Thus, at “steady-state,” the patient's body eliminates the drug at approximately the same rate that the drug becomes available to the patient's system through absorption into the blood stream.

The term “treating” or “treatment” as used herein refers to relieving, reducing, managing, or attenuating the pain associated with irritable bowel syndrome.

The term “preventing” or “prevent” as used herein refers to delaying onset of and/or decreasing the risk of developing pain associated with irritable bowel syndrome.

The term “manage” or “managing” as used herein refers to providing beneficial effects to a patient, which does not result in a cure.

The term “pain” as used herein refers to any disagreeable sensory experience and includes visceral pain and functional pain.

The term “pain associated with IBS” as used herein refers to any pain in the upper, mid and/or lower abdominal area associated with IBS. Pain associated with IBS may be caused by a variety of factors, which may include colon spasm, abdominal distention (constipation or bloating), and/or peripheral and/or central sensitization.

The term “subject” or “patient” as used herein refers to someone who is at a heightened risk of suffering from, is currently suffering from, or has at any time in the past suffered from pain associated with IBS. A subject is at a heightened risk of suffering from pain associated with IBS, for example, when the subject has developed IBS symptoms unrelated to pain or when pain associated with IBS has been relieved, reduced, managed, or attenuated previously in the subject.

In some embodiments, the method of treating or preventing pain associated with IBS comprises orally administering to a subject in need thereof vibegron over a treatment period. The term “treatment period” means the period of time during which the drug is administered to a subject. For example, the treatment period can be from about 2 weeks to about 2 years. In some embodiments, the treatment period can be about 2, about 4, about 6, about 8, about 10, about 12, about 14, about 16, about 18, about 20, about 24, about 52, about 76 or about 104 weeks. In some embodiments, the treatment period can be greater than about 2, about 4, about 6, about 8, about 10, about 12, about 14, about 16, about 18, about 20, about 24, about 52, about 76 or about 104 weeks. The efficacy of the drug can be assessed by measuring certain parameters and calculating the changes from baseline over the treatment period.

Additional definitions related to irritable bowel syndrome and chronic idiopathic constipation can be found, for example, in Ford et al. (2014) “American College of Gastroenterology Monograph on the Management of Irritable Bowel Syndrome and Chronic Idiopathic Constipation” Am J Gastroenterol 109:S2-S26. All these documents are herein incorporated by reference in their entireties.

### Methods of Treatment

The present disclosure relates to a method of treating or preventing pain associated with IBS comprising orally administering to a subject in need thereof a dosage of vibegron such that the desired efficacy is maintained while the undesirable side effects are minimized. Without wishing to be bound by theory, vibegron is considered to provide improvement in abdominal pain due to IBS in both male and female patients. The effect of beta-3 agonists on IBS-related abdominal pain in males has varied.

Again without wishing to be bound by theory, it is hypothesized that vibegron can reduce colon smooth muscle contractions, enteric neuron hyperactivity and visceral pain by mediating a direct effect on smooth muscle and indirectly by promoting release of somatostatin (SST) within the intestine. Therefore, by direct action on colon spasm and indirectly through SST, vibegron can reduce abdominal pain in patients with IBS, including IBS-D, IBS-M, IBS-C, and IBS-U subtypes. Thus, unlike solabegron, which showed variable efficacy in different IBS subtype populations, worsening of IBS symptoms, less potency in pharmacologic activity on beta-3 receptors, and a shorter duration of effect, vibegron can treat pain associated with IBS across subtypes IBS-D, IBS-M, IBS-C, and/or IBS-U subtypes.

In addition, without wishing to be bound by theory, vibegron is considered to provide an alteration in gastrointestinal or colonic transit. Effect on transit time is found to vary between beta-3 agonists.

The present disclosure provides a method of treating pain associated with IBS, the method comprising orally administering to a subject in need thereof an amount of from about 1 mg to about 1000 mg of vibegron per day. In another aspect, the present disclosure provides a method of preventing pain associated with IBS, the method comprising orally administering to a subject in need thereof an amount of from about 1 mg to about 1000 mg of vibegron per day.

In some embodiments, the amount of vibegron administered per day is from about 2 mg to about 1000 mg, from about 3 mg to about 1000 mg, from about 4 mg to about 1000 mg, from about 5 mg to about 1000 mg, from about 10 mg to about 1000 mg, from about 15 mg to about 1000 mg, from about 20 mg to about 1000 mg, from about 25 mg to about 1000 mg, from about 30 mg to about 1000 mg, from about 40 mg to about 1000 mg, from about 50 mg to about 1000 mg, from about 60 mg to about 1000 mg, from about 70 mg to about 1000 mg, from about 80 mg to about 1000 mg, from about 90 mg to about 1000 mg, from about 100 mg to about 1000 mg, from about 150 mg to about 1000 mg, from about 200 mg to about 1000 mg, from about 250 mg to about 1000 mg, from about 300 mg to about 1000 mg, from about 350 mg to about 1000 mg, from about 400 mg to about 1000 mg, from about 450 mg to about 1000 mg, from about 500 mg to about 1000 mg, from about 550 mg to about 1000 mg, from about 600 mg to about 1000 mg, from about 650 mg to about 1000 mg, from about 700 mg to about 1000 mg, from about 750 mg to about 1000 mg, from about 800 mg to about 1000 mg, from about 850 mg to about 1000 mg, from about 900 mg to about 1000 mg, or from about 950 mg to about 1000 mg.

In some embodiments, the amount of vibegron administered per day is from about 1 mg to about 950 mg, from about 1 mg to about 900 mg, from about 1 mg to about 850 mg, from about 1 mg to about 800 mg, from about 1 mg to about 750 mg, from about 1 mg to about 700 mg, from about 1 mg to about 650 mg, from about 1 mg to about 600 mg, from about 1 mg to about 550 mg, from about 1 mg to about 500 mg, from about 1 mg to about 450 mg, from about 1 mg to about 400 mg, from about 1 mg to about 350 mg, from about 1 mg to about 300 mg, from about 1 mg to about 250 mg, from about 1 mg to about 200 mg, from about 1 mg to about 150 mg, from about 1 mg to about 100 mg, from about 1 mg to about 90 mg, from about 1 mg to about 80 mg, from about 1 mg to about 70 mg, from about 1 mg to about 60 mg, from about 1 mg to about 50 mg, from about 1 mg to about 40 mg, from about 1 mg to about 30 mg, from about 1 mg to about 25 mg, from about 1 mg to about 20 mg, from about 1 mg to about 15 mg, from about 1 mg to about 10 mg, from about 1 mg to about 5 mg, from about 1 mg to about 4 mg, from about 1 mg to about 3 mg, or from about 1 mg to about 2 mg.

In some embodiments, the present disclosure provides a method of treating or preventing pain associated with IBS, the method comprising orally administering to a subject in need thereof an amount of from 25 mg to 150 mg of vibegron per day.

In some embodiments, the amount of vibegron administered per day is from about 30 mg to about 145 mg, about 35 mg to about 140 mg, about 40 mg to about 135 mg, about 45 mg to about 130 mg, about 50 mg to about 125 mg, about 55 mg to about 120 mg, about 60 mg to about 115 mg, about 65 mg to about 110 mg, about 70 mg to about 105 mg, about 75 mg to about 100 mg, about 80 mg to about 95 mg, or about 85 mg to about 90 mg.

In some embodiments, the amount of vibegron administered per day is from about 25 mg to about 145 mg, about 25 mg to about 140 mg, about 25 mg to about 135 mg, about 25 mg to about 130 mg, about 25 mg to about 125 mg, about 25 mg to about 120 mg, about 25 mg to about 115 mg, about 25 mg to about 110 mg, about 25 mg to about 105 mg, about 25 mg to about 100 mg, about 25 mg to about 95 mg, about 25 mg to about 90 mg, about 25 mg to about 85 mg, about 25 mg to about 80 mg, about 25 mg to about 75 mg, about 25 mg to about 70 mg, about 25 mg to about 65 mg, about 25 mg to about 60 mg, about 25 mg to about 55 mg, about 25 mg to about 50 mg, about 25 mg to about 45 mg, about 25 mg to about 40 mg, about 25 mg to about 35 mg, or about 25 mg to about 30 mg.

In some embodiments, the amount of vibegron administered per day is from about 30 mg to about 150 mg, about 35 mg to about 150 mg, about 40 mg to about 150 mg, about 45 mg to about 150 mg, about 50 mg to about 150 mg, about 55 mg to about 150 mg, about 60 mg to about 150 mg, about 65 mg to about 150 mg, about 70 mg to about 150 mg, about 75 mg to about 150 mg, about 80 mg to about 150 mg, about 85 mg to about 150 mg, about 90 mg to about 150 mg, about 95 mg to about 150 mg, about 100 mg to about 150 mg, about 105 mg to about 150 mg, about 110 mg to about 150 mg, about 115 mg to about 150 mg, about 120 mg to about 150 mg, about 125 mg to about 150 mg, about 130 mg to about 150 mg, about 135 mg to about 150 mg, about 140 mg to about 150 mg, or about 145 mg to about 150 mg.

In some embodiments, the amount of vibegron administered per day is from about 55 mg to about 100 mg, from about 60 mg to about 100 mg, from about 65 mg to about 100 mg, from about 70 mg to about 100 mg, from about 75 mg to about 100 mg, from about 80 mg to about 100 mg, from about 85 mg to about 100 mg, from about 90 mg to about 100 mg, or from about 95 mg to about 100 mg.

In some embodiments, the amount of vibegron administered per day is from about 50 mg to about 95 mg, from about 50 mg to about 90 mg, from about 50 mg to about 85 mg, from about 50 mg to about 80 mg, from about 50 mg to about 75 mg, from about 50 mg to about 70 mg, from about 50 mg to about 65 mg, from about 50 mg to about 60 mg, or from about 50 mg to about 55 mg.

In some embodiments, the amount of vibegron administered per day is from about 60 mg to about 90 mg, from about 65 mg to about 85 mg, or from about 70 mg to about 80 mg. In some embodiments, the amount of vibegron administered per day is from 60 mg to 90 mg, from 65 mg to 85 mg, or from 70 mg to 80 mg.

In some embodiments, the amount of vibegron administered per day is about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg. In some embodiments, the amount of vibegron administered per day is about 50 mg. In some embodiments, the amount of vibegron administered per day is 50 mg. In some embodiments, the amount of vibegron administered per day is about 75 mg. In some embodiments, the amount of vibegron administered per day is 75 mg. In some embodiments, the amount of vibegron administered per day is about 100 mg. In some embodiments, the amount of vibegron administered per day is 100 mg.

In some embodiments, the present disclosure provides a method of treating pain associated with IBS, the method comprising orally administering to a subject in need thereof about 75 mg of vibegron per day.

In some embodiments, the amount of vibegron administered per day is not about 50 mg. In some embodiments, the amount of vibegron administered per day is not about 75 mg. In some embodiments, the amount of vibegron administered per day is not about 100 mg. In some embodiments, the amount of vibegron administered per day is not 50 mg. In some embodiments, the amount of vibegron administered per day is not 75 mg. In some embodiments, the amount of vibegron administered per day is not 100 mg.

In some embodiments, the subject has a symptom selected from the group consisting of abnormal stool frequency, greater than 3 bowel movements per day, less than 3 bowel movements per week, abnormal stool form or consistency (lumpy/hard or loose/watery stool), abnormal stool passage (straining, urgency, or feeling of incomplete evacuation), abnormal urgency, passage of mucus, bloating or feeling of abdominal distension, abdominal discomfort, bowel movements with incomplete evacuation, and combinations thereof. In these embodiments, the subject experiences an improvement in one or more of these symptoms after treatment with about 75 mg of vibegron per day, as discussed below.

In some embodiments, the subject experiences an improvement in abnormal stool frequency after treatment with vibegron. In some embodiments, a subject with IBS-D experiences a decrease in abnormal stool frequency after treatment with vibegron. In some embodiments, a subject with IBS-D has fewer than 3 bowel movements per day after treatment with vibegron. In some embodiments, a subject with IBS-C experiences an increase in stool frequency after treatment with vibegron. In some embodiments, a subject with IBS-C experiences more than 3 bowel movements per week after treatment with vibegron. As IBS-M is characterized by episodes of diarrhea and constipation, in some embodiments a subject with IBS-M after treatment with vibegron can experience an increase in stool frequency relative to that seen in periods of constipation and/or can experience a decrease in stool frequency relative to that seen in periods of diarrhea. In some embodiments, a subject with IBS-U can experience an increase in stool frequency or a decrease in stool frequency after treatment with vibegron.

In some embodiments, the subject experiences an improvement in abnormal stool form or consistency after treatment with vibegron. In some embodiments, a subject with IBS-D experiences a reduction in loose/watery stool after treatment with vibegron. In some embodiments, a subject with IBS-D experiences a decrease in score on the Bristol Stool Form Scale after treatment with vibegron. In some embodiments, a subject with IBS-D experiences a decrease in score of from about 2 points to about 5 points, from about 2 points to about 4 points, from about 2 to about 3 points, from about 1 point to about 3 points, or from about 1 point to about 2 points on the Bristol Stool Form Scale after treatment with vibegron. In some embodiments, a subject with IBS-C experiences a reduction in lumpy/hard stool after treatment with vibegron. In some embodiments, a subject with IBS-C experiences an increase in score on the Bristol Stool Form Scale after treatment with vibegron. In some embodiments, a subject with IBS-C experiences an increase in score of from about 1 point to about 4 points, from about 1 to about 3 points, from about 1 to about 2 points, from about 2 to about 3 points, or from about 2 to about 4 points on the Bristol Stool Form Scale after treatment with vibegron. As IBS-M is characterized by episodes of diarrhea and constipation, in some embodiments a subject with IBS-M after treatment with vibegron can experience an increase in score on the Bristol Stool Form Scale relative to that seen in periods of constipation, and/or can experience a decrease in score on the Bristol Stool Form Scale relative to that seen during periods of diarrhea. In some embodiments, the increase or decrease in score on the Bristol Stool Form Scale is as above described for IBS-C or IBS-D respectively. In some embodiments, a subject with IBS-U after treatment with vibegron can experience an increase or a decrease in score on the Bristol Stool Form Scale. In some embodiments, the increase or decrease in score on the Bristol Stool Form Scale is as above described for IBS-C or IBS-D respectively.

In some embodiments, the subject experiences an improvement in abnormal stool passage after treatment with vibegron. In some embodiments, a subject with IBS-C experiences a decrease in straining during bowel movements after treatment with vibegron. In some embodiments, a subject with IBS-C experiences a decrease in bowel movements with incomplete evacuation after treatment with vibegron. In some embodiments, a subject with IBS-D experiences a decrease in sense of urgency after treatment with vibegron. As IBS-M is characterized by episodes of diarrhea and constipation, in some embodiments a subject with IBS-M after treatment with vibegron can experience a decrease in straining during bowel movements and/or a decrease in bowel movements with incomplete evacuation, and/or a decrease in sense of urgency. In some embodiments, a subject with IBS-U after treatment with vibegron can experience a decrease in straining during bowel movements and/or a decrease in feeling of incomplete evacuation, and/or a decrease in sense of urgency.

In some embodiments, after treatment with vibegron the subject experiences a decrease in passage of mucus, bloating, and/or feelings of abdominal distension. In some embodiments, after treatment with vibegron a subject with IBS-D experiences a decrease in passage of mucus, bloating and/or feelings of abdominal distension. As IBS-M is characterized by episodes of diarrhea and constipation, in some embodiments a subject with IBS-M after treatment with vibegron can experience a decrease in passage of mucus, bloating, and/or feelings of abdominal distension. In some embodiments, a subject with IBS-U after treatment with vibegron can experience a decrease in passage of mucus, bloating, and/or feelings of abdominal distension. In some embodiments, the subject experiences an improvement in abdominal discomfort after treatment with vibegron. In some embodiments, a subject with IBS-D, IBS-C, IBS-M, or IBS-U experiences an improvement in abdominal discomfort after treatment with vibegron.

In some embodiments, the subject experiences a combination of any of the above improvements after treatment with vibegron. In some embodiments, a subject with IBS-D, IBS-C, IBS-M, or IBS-U experiences a combination of any of the above improvements after treatment with vibegron.

In some embodiments, the subject has a symptom of abdominal discomfort that has one or more of the following features: relieved with defecation; and/or onset associated with a change in frequency of stool; and/or onset associated with a change in form (appearance) of stool. In some embodiments, the subject has an improvement in symptoms of abdominal discomfort that have one or more of the following features: relieved with defecation; and/or onset associated with a change in frequency of stool; and/or onset associated with a change in form (appearance) of stool. In some embodiments, a subject with IBS-D, IBS-C, IBS-M, or IBS-U has an improvement in symptoms of abdominal discomfort that have one or more of the following features: relieved with defecation; and/or onset associated with a change in frequency of stool; and/or onset associated with a change in form (appearance) of stool.

In some embodiments, the subject suffers from IBS-C, IBS-D, IBS-M, and/or IBS-U. In some embodiments, the subject suffers from IBS-D. In some embodiments, the subject suffers from IBS-C. In some embodiment, the subject suffers from IBS-M. In some embodiment, the subject suffers from IBS-U. In some embodiments, the subject suffers from IBS-D and/or IBS-M.

In some embodiments, the subject is at a heightened risk of experiencing pain associated with IBS. In some embodiments, the subject is not experiencing pain associated with IBS.

In some embodiments, the subject is a mammal. In some embodiments the subject is a human or an animal. In some embodiments, the subject is a human. In some embodiments the subject is a female. In some embodiments the subject is a male. In some embodiments the subject is a juvenile.

In some embodiments, the subject is over the age of about 18 years. In some embodiments, the subject is under the age of about 18 years. In some embodiments the subject is over the age of about 20 years. In some embodiments the subject is over the age of about 25 years. In some embodiments the subject is over the age of about 30 years. In some embodiments the subject is over the age of about 35 years. In some embodiments the subject is over the age of about 40 years. In some embodiments, the subject is over the age of about 45 years. In some embodiments, the subject is over the age of about 50 years. In some embodiments, the subject is over the age of about 55 years. In some embodiments, the subject is over the age of about 60 years. In some embodiments, the subject is over the age of about 65 years. In some embodiments, the subject is over the age of about 70 years. In some embodiments, the subject is over the age of about 75 years.

In some embodiments the subject is between the age of about 18 years to about 50 years. In some embodiments the subject is between the age of about 20 years to about 50 years. In some embodiments the subject is between the age of about 25 years to about 50 years. In some embodiments the subject is between the age of about 30 years to about 50 years. In some embodiments the subject is between the age of about 35 years to about 50 years. In some embodiments the subject is between the age of about 40 years to about 50 years. In some embodiments the subject is a female between the age of about 18 years to about 50 years.

In some embodiments, the subject is between the age of about 6 years to about 18 years. In some embodiments, the subject is between the age of about 6 years to about 12 years. In some embodiments, the subject is between the age of about 12 years to 18 years.

In some embodiments, the subject has received prior IBS therapy, to treat IBS and/or one or more symptoms, features, or manifestations thereof. In some embodiments, the subject has not received prior IBS therapy, to treat IBS and/or one or more symptoms, features, or manifestations thereof.

In some embodiments the subject suffers from renal impairment and is administered about 50, 75, or 100 mg of vibegron per day.

In some embodiments, the subject is concomitantly receiving, taking or otherwise being exposed to at least one additional therapeutic agent, wherein the therapeutic agent is selected from a therapeutic agent for irritable bowel syndrome, a therapeutic agent for diarrhea, a therapeutic agent for constipation, an ameliorating agent for abdominal pain, a digestive tract motility regulator, a digestive tract motility activator, an anti-depressant agent, an anti-anxiety agent, and combinations thereof.

In some embodiments, the subject is concomitantly receiving, taking or otherwise being exposed to at least one additional therapeutic agent, wherein the therapeutic agent is selected from a therapeutic agent for IBS-C, IBS-D, IBS-M, and/or IBS-U.

In some embodiments, the subject is not concomitantly receiving, taking or otherwise being exposed to at least one additional therapeutic agent, wherein the therapeutic agent is selected from a therapeutic agent for irritable bowel syndrome, a therapeutic agent for diarrhea, a therapeutic agent for constipation, an ameliorating agent for abdominal pain, a digestive tract motility regulator, a digestive tract motility activator, an anti-depressant agent, an anti-anxiety agent, and combinations thereof

In some embodiments, the subject is eating a diet restricted in fermentable oligo-, di-, monosaccharides and polyols (FODMAPs). In some embodiments the subject is receiving, taking or otherwise being exposed to soluble fiber supplementation.

In some embodiments the subject is concomitantly receiving, taking or otherwise being exposed to a guanylate cyclase 2C agonist.

In some embodiment the subject is concomitantly receiving, taking or otherwise being exposed to linaclotide.

In some embodiment the subject is concomitantly receiving, taking or otherwise being exposed to plecanatide.

In some embodiments the subject is concomitantly receiving, taking or otherwise being exposed to an μ- and/or κ-opioid receptor agonist.

In some embodiments the subject is concomitantly receiving, taking or otherwise being exposed to an δ-opioid receptor antagonist.

In some embodiment the subject is concomitantly receiving, taking or otherwise being exposed to eluxadoline.

In some embodiments the subject is concomitantly receiving, taking or otherwise being exposed to ClC-2 chloride channel activator.

In some embodiment the subject is concomitantly receiving, taking or otherwise being exposed to lubiprostone.

In some embodiments the subject is concomitantly receiving, taking or otherwise being exposed to an antibiotic.

In some embodiment the subject is concomitantly receiving, taking or otherwise being exposed to rifaximin.

In some embodiment the subject is concomitantly receiving, taking, or otherwise being exposed to a cytochrome P450 inhibitor, such as a CYP3A inhibitor, and with drugs that are substrates of the following CYPs: CYP1A2, 2B6, 2C8, 2C9, 2C19, 2D6, and 3 A4.

In some embodiments, the subject is concomitantly receiving, taking, or otherwise being exposed to a P-glycoprotein inhibitor.

CYP3A/P-glycoprotein inhibitors include but are not limited to amiodarone, carvedilol, clarithromycin, dronedarone, itraconazole, lapatinib, lopinavir and ritonavir, propafenone, quinidine, ranolazine, ritonavir, saquinavir and ritonavir, telaprevir, tipranavir and ritonavir, verapamil, curcumin, cyclosporine A, eltrombopag, atazanavir and ritonavir, clarithromycin, cyclosporine, erythromycin, gemfibrozil, rifampin (single dose), simeprevir, p-aminohippuric acid (PAH)(b), probenecid, teriflunomide, cimetidine, dolutegravir, isavuconazole, ranolazine, trimethoprim, and vandetanib.

In some embodiments, the subject is administered about 50, 75, or 100 mg of vibegron per day and is concomitantly receiving, taking or otherwise being exposed to a CYP3A inhibitor.

In some embodiments, the subject is administered about 50, 75, or 100 mg of vibegron per day and is concomitantly receiving, taking, or otherwise being exposed to a P-glycoprotein inhibitor.

In some embodiments, the subject is administered about 50, 75, or 100 mg of vibegron per day and is concomitantly receiving, taking or otherwise being exposed to a guanylate cyclase 2C agonist.

In some embodiments, the subject is administered about 50, 75, or 100 mg of vibegron per day and is concomitantly receiving, taking, or otherwise being exposed to a μ- and/or κ-opioid receptor agonist.

In some embodiments the subject is not concomitantly receiving, taking, or otherwise being exposed to a beta blocker.

In some embodiments the subject is not concomitantly receiving, taking, or otherwise being exposed to an amlodipine.

In some embodiments, vibegron is administered with a meal. In some embodiments, vibegron is administered after a meal. In some embodiment, vibegron is administered within 60 minutes after a meal, within 2 hours after a meal, or within 3 hours after a meal.

In some embodiments, vibegron is administered without a meal. In some embodiments, vibegron is administered before a meal. In some embodiments, vibegron is administered more than three hours before a meal, more than two hours before a meal, or more than 60 minutes before a meal.

In some embodiments, vibegron is administered once per day, twice per day, or three times per day. In some embodiments, vibegron is administered once per day. In some embodiments vibegron is administered twice per day.

In some embodiments, the subject experiences an increase in stool frequency over the treatment period. In some embodiments, the subject experiences a decrease in stool frequency over the treatment period.

In some embodiments, the subject experiences a change in stool consistency over the treatment period. In some embodiments the subject does not experience a change in stool consistency over the treatment period. In some embodiments the change is measured according to the Bristol Form Scale.

In some embodiments, the subject experiences a change in gastrointestinal or colonic transit. Significant alterations in these transit measurements have proven to be predictive of efficacy in phase IIB or phase III clinical trials. In some embodiments, gastrointestinal transit can be measured with scintigraphy. In this technique, a methacrylate-coated, delayed-release capsule containing 0.1 mCi of ¹¹¹InCl₃ absorbed on activated charcoal particles is ingested. Two hours after ingestion, two scrambled eggs labeled with ^{99m}Tc-sulfur colloid are ingested with one slice of whole wheat bread and one glass of skim milk. Anterior and posterior gamma camera images are then obtained at 0, 1, 2, 4, 6, 24, and 48 hours after radioactive meal ingestion. The primary outcome variables include the percentage of radioisotope emptied from the stomach at 1, 2, and 4 hours, the gastric half-emptying time from linear interpolation of the gastric residuals, the percentage of colonic filing at 6 hours, and the colonic geometric center (GC) at 4, 8, 24, and 48 hours.

In some embodiments, a subject with IBS-D experiences a decrease in gastrointestinal and/or colonic transit after treatment with vibegron. In some embodiments, a subject with IBS-C experiences an increase in gastrointestinal and/or colonic transit after treatment with vibegron. As IBS-M is characterized by periods of diarrhea and constipation, in some embodiments after treatment with vibegron a subject with IBS-M experiences a decrease in gastrointestinal and/or colonic transit relative to that during periods of diarrhea and/or an increase in colonic transit relative to that during periods of constipation. In some embodiments, a subject with IBS-U experiences an increase or decrease in gastrointestinal and/or colonic transit after treatment with vibegron. In these embodiments, the subject is administered about 75 mg of vibegron per day.

In some embodiments, the subject experiences a change in somatostatin plasma concentration. In some embodiments the subject experiences an increase in somatostatin.

In some embodiments, the subject does not experience an increase in an adverse event selected from nasopharyngitis, headache, nausea, gastroenteritis, IBS (such as diarrhea, constipation, or both), and a combination thereof.

In some embodiments, the subject experiences a decrease in the average number of daily urgency episodes over the treatment period. In some embodiments the subject experiences an increase in the average number of daily urgency episodes over the treatment period.

In some embodiments, the subject experiences a decrease in weekly number of days with recurrent bowel movements. In some embodiments the subject experiences an increase in the weekly number of days with recurrent bowel movements.

Efficacy of the method of treatment disclosed herein can be determined based on the subject's response to a daily question, for example, “In regards to your specific IBS symptom of abdominal pain, on a scale of 0-10, what was your worst IBS-related abdominal pain in the last 24 hours? ‘Zero’ means you have no pain at all; ‘Ten’ means the worst possible pain you can imagine.” In some embodiments, weekly treatment success in IBS-related pain is defined as a 30% or greater improvement from baseline in the weekly average abdominal pain score, based on subject response to a daily question. For example, the weekly average abdominal pain score can be a weekly average of “worst abdominal pain in the past 24 hours” score on a 0 to 10 point numeric rating scale (NRS).

The treatment efficacy can be measured by the abdominal pain intensity (API) weekly responder rate at the end of the treatment period. An API Weekly Responder is defined as a subject who experiences a decrease in the weekly average of “worst abdominal pain in the past 24 hours” scores of at least 30% compared with the baseline weekly average.

In some embodiments the subject is experiencing a weekly average of “worst abdominal pain in the past 24 hours” scores of less than or equal to 2 before treatment.

In some embodiments the subject is experiencing a weekly average of “worst abdominal pain in the past 24 hours” scores of less than or equal to 4 before treatment.

In some embodiments the subject is experiencing a weekly average of “worst abdominal pain in the past 24 hours” scores of less than or equal to 6 before treatment.

In some embodiments the subject is experiencing a weekly average of “worst abdominal pain in the past 24 hours” scores of greater than or equal to 6 before treatment.

In some embodiments the subject is experiencing a weekly average of “worst abdominal pain in the past 24 hours” scores of greater than or equal to 8 before treatment.

In some embodiments, the subject does not experience an increase in the weekly average abdominal pain score compared with baseline. In some embodiments, the subject experiences an increase of less than about 10 percent compared with baseline. In some embodiments, the subject experiences an increase of less than about 20 percent compared with baseline. In some embodiments, the subject experiences an increase of less than about 30 percent compared with baseline. In some embodiments, the subject experiences an increase of less than about 40 percent compared with baseline. In some embodiments, the subject experiences an increase of less than about 50 percent compared with baseline. In some embodiments, the subject experiences an increase of less than about 60 percent compared with baseline. In some embodiments, the subject experiences an increase of less than about 70 percent compared with baseline. In some embodiments, the subject experiences an increase of less than about 80 percent compared with baseline. In some embodiments, the subject experiences an increase of less than about 90 percent compared with baseline.

In some embodiments, the subject experiences a decrease in the weekly average abdominal pain score compared with baseline. In some embodiments, the subject experiences a decrease of at least about 30 percent compared with baseline. In some embodiments, the subject experiences a decrease of at least about 40 percent compared with baseline. In some embodiments, the subject experiences a decrease of at least about 50 percent compared with baseline. In some embodiments, the subject experiences a decrease of at least about 60 percent compared with baseline. In some embodiments, the subject experiences a decrease of at least about 70 percent compared with baseline. In some embodiments, the subject experiences a decrease of at least about 80 percent compared with baseline. In some embodiments, the subject experiences a decrease of at least about 90 percent compared with baseline. In some embodiments, the subject experiences a decrease of about 100 percent compared with baseline.

The treatment efficacy can be measured by IBS-Quality of Life (QoL) scores. An IBS-QoL responder is defined as a subject who has achieved at least a 14-point improvement in IBS-QoL total score. The IBS-QoL questionnaire consists of 34 items, each with a five-point response scale.

In some embodiments, the subject achieves at least a 15-point, at least a 16-point, at least a 17-point, at least an 18-point, at least a 19-point, or at least a 20-point improvement in IBS-QoL total score.

The treatment efficacy can also be measured by the Mean Patient Global Impression Scale (GIS) score at the end of the treatment period.

“Global Impression Scale” is also known as “Global Improvement Scale.” Global improvement assessment asks subjects to evaluate their current IBS status by asking the following question: How would you rate your IBS signs or symptoms overall over the past 7 days?
1) significantly relieved,
2) moderately relieved,
3) slightly relieved,
4) unchanged,
5) slightly worse,
6) moderately worse,
7) significantly worse.

A GIS responder is defined as a subject who answered that their symptoms were either moderately relieved or significantly relieved.

In some embodiments, the measurement of treatment efficacy discussed herein can be based on the change from baseline over the treatment period. In some embodiments, the measurement of treatment efficacy discussed herein can be based on the change from baseline over the treatment period as compared to that in the subjects taking placebo.

In some embodiments, the treatment period begins before the subject has experienced pain associated with IBS.

Changes from baseline in blood pressure (BP) and heart rate (HR) for the subjects taking vibegron are not substantially different for the subjects taking a placebo. In some embodiments, the subject experiences a mean maximum change of systolic blood pressure (SBP) from baseline over the treatment period (e.g., 8 weeks or 12 weeks), and the mean maximum change is less than 2.0 mm/Hg, less than 1.9 mm/Hg, less than 1.8 mm/Hg, less than 1.7 mm/Hg, less than 1.6 mm/Hg, less than 1.5 mm/Hg, less than 1.4 mm/Hg, less than 1.3 mm/Hg, less than 1.2 mm/Hg, less than 1.1 mm/Hg, less than 1.0 mm/Hg, less than 0.9 mm/Hg, less than 0.8 mm/Hg, less than 0.7 mm/Hg, less than 0.6 mm/Hg, or less than 0.5 mm/Hg from that of a subject taking a placebo.

In some embodiments, the subject is administered about 75 mg of vibegron per day, and experiences a mean maximum change of SBP from baseline over the treatment period (e.g., 8 weeks or 12 weeks) of less than 2 mm/Hg from that of a subject taking a placebo. In some embodiments, the subject is administered about 75 mg of vibegron per day, and experiences a mean maximum change of SBP from baseline over the treatment period (e.g., 8 weeks or 12 weeks) of less than 1 mm/Hg from that of a subject taking a placebo.

In some embodiments, the subject is female and is administered about 75 mg of vibegron per day, and experiences a mean maximum change of SBP from baseline over the treatment period (e.g., 8 weeks or 12 weeks) of less than 2 mm/Hg from that of a subject taking a placebo. In some embodiments, the subject is female and is administered about 75 mg of vibegron per day, and experiences a mean maximum change of SBP from baseline over the treatment period (e.g., 8 weeks or 12 weeks) of less than 1 mm/Hg from that of a subject taking a placebo.

In some embodiments, the subject is male and is administered about 75 mg of vibegron per day, and experiences a mean maximum change of SBP from baseline over the treatment period (e.g., 8 weeks or 12 weeks) of less than 2 mm/Hg from that of a subject taking a placebo. In some embodiments, the subject is male and is administered about 75 mg of vibegron per day, and experiences a mean maximum change of SBP from baseline over the treatment period (e.g., 8 weeks or 12 weeks) of less than 1 mm/Hg from that of a subject taking a placebo.

In some embodiments, the subject experiences a mean maximum change of diastolic blood pressure (DBP) from baseline over the treatment period (e.g., 8 weeks or 12 weeks), and the mean maximum change is less than 2.0 mm/Hg, less than 1.9 mm/Hg, less than 1.8 mm/Hg, less than 1.7 mm/Hg, less than 1.6 mm/Hg, less than 1.5 mm/Hg, less than 1.4 mm/Hg, less than 1.3 mm/Hg, less than 1.2 mm/Hg, less than 1.1 mm/Hg, less than 1.0 mm/Hg, less than 0.9 mm/Hg, less than 0.8 mm/Hg, less than 0.7 mm/Hg, less than 0.6 mm/Hg, or less than 0.5 mm/Hg from that of a subject taking a placebo.

In some embodiments, the subject is administered about 75 mg of vibegron per day, and experiences a mean maximum change of DBP from baseline over the treatment period (e.g., 8 weeks or 12 weeks) of less than 2 mm/Hg from that of a subject taking a placebo. In some embodiments, the subject is administered about 75 mg of vibegron per day, and experiences a mean maximum change of DBP from baseline over the treatment period (e.g., 8 weeks or 12 weeks) of less than 1 mm/Hg from that of a subject taking a placebo.

In some embodiments, the subject is female and is administered about 75 mg of vibegron per day, and experiences a mean maximum change of DBP from baseline over the treatment period (e.g., 8 weeks or 12 weeks) of less than 2 mm/Hg from that of a subject taking a placebo. In some embodiments, the subject is female and is administered about 75 mg of vibegron per day, and experiences a mean maximum change of DBP from baseline over the treatment period (e.g., 8 weeks or 12 weeks) of less than 1 mm/Hg from that of a subject taking a placebo.

In some embodiments, the subject is male and is administered about 75 mg of vibegron per day, and experiences a mean maximum change of DBP from baseline over the treatment period (e.g., 8 weeks or 12 weeks) of less than 2 mm/Hg from that of a subject taking a placebo. In some embodiments, the subject is male and is administered about 75 mg of vibegron per day, and experiences a mean maximum change of DBP from baseline over the treatment period (e.g., 8 weeks or 12 weeks) of less than 1 mm/Hg from that of a subject taking a placebo.

In some embodiments, the subject is male, is administered about 75 mg of vibegron once per day, and experiences a mean maximum change of DBP from baseline over the treatment period (e.g., 8 weeks or 12 weeks) of less than 1 mm/Hg from that of a subject taking a placebo, and a mean maximum change of SBP from baseline over the treatment period (e.g., 8 weeks or 12 weeks) of less than 1 mm/Hg from that of a subject taking a placebo.

In some embodiments, the subject is female, is administered about 75 mg of vibegron once per day, and experiences a mean maximum change of DBP from baseline over the treatment period (e.g., 8 weeks or 12 weeks) of less than 1 mm/Hg from that of a subject taking a placebo, and a mean maximum change of SBP from baseline over the treatment period (e.g., 8 weeks or 12 weeks) of less than 1 mm/Hg from that of a subject taking a placebo.

In some embodiments, the subject experiences a mean maximum change of systolic blood pressure (SBP) from baseline over the treatment period (e.g., 8 weeks or 12 weeks) of less than 10 mm/Hg, less than 9.5 mm/Hg, less than 9 mm/Hg, less than 8.5 mm/Hg, less than 8 mm/Hg, less than 7.5 mm/Hg, less than 7 mm/Hg, less than 6.5 mm/Hg, less than 6 mm/Hg, less than 5.5 mm/Hg, or less than 5 mm/Hg.

In some embodiments, the subject is administered about 75 mg of vibegron per day, and experiences a mean maximum change of SBP from baseline over the treatment period (e.g., 8 weeks or 12 weeks) of less than 10 mm/Hg.

In some embodiments, the subject is female and is administered about 75 mg of vibegron per day, and experiences a mean maximum change of SBP from baseline over the treatment period (e.g., 8 weeks or 12 weeks) of less than 10 mm/Hg.

In some embodiments, the subject is male and is administered about 75 mg of vibegron per day, and experiences a mean maximum change of SBP from baseline over the treatment period (e.g., 8 weeks or 12 weeks) of less than 10 mm/Hg.

In some embodiments, the subject experiences a mean maximum change of diastolic blood pressure (DBP) from baseline over the treatment period (e.g., 8 weeks or 12 weeks) of less than 7 mm/Hg, less than 6.5 mm/Hg, less than 6 mm/Hg, less than 5.5 mm/Hg, less than 5 mm/Hg, less than 4.5 mm/Hg, less than 4 mm/Hg, less than 3.5 mm/Hg, less than 3 mm/Hg, less than 2.5 mm/Hg, or less than 2 mm/Hg.

In some embodiments, the subject is administered about 75 mg of vibegron per day, and experiences a mean maximum change of DBP from baseline over the treatment period (e.g., 8 weeks or 12 weeks) of less than 7 mm/Hg.

In some embodiments, the subject is female and is administered about 75 mg of vibegron per day, and experiences a mean maximum change of DBP from baseline over the treatment period (e.g., 8 weeks or 12 weeks) of less than 7 mm/Hg.

In some embodiments, the subject is male and is administered about 75 mg of vibegron per day, and experiences a mean maximum change of DBP from baseline over the treatment period (e.g., 8 weeks or 12 weeks) of less than 7 mm/Hg.

In some embodiments, the subject male is administered about 75 mg of vibegron once per day, experiences a mean maximum change of DBP from baseline over the treatment period (e.g., 8 weeks or 12 weeks) of less than 7 mm/Hg, and a mean maximum change of SBP from baseline over the treatment period (e.g., 8 weeks or 12 weeks) of less than 10 mm/Hg.

In some embodiments, the subject female is administered about 75 mg of vibegron once per day, experiences a mean maximum change of DBP from baseline over the treatment period (e.g., 8 weeks or 12 weeks) of less than 7 mm/Hg, and a mean maximum change of SBP from baseline over the treatment period (e.g., 8 weeks or 12 weeks) of less than 10 mm/Hg.

### Pharmaceutical Unit Dose Composition

The present disclosure provides pharmaceutical unit dose compositions comprising a dosage of vibegron disclosed herein, wherein the unit dosage composition is suitable for oral administration. Oral dosage forms are recognized by those skilled in the art to include, for example, such forms as liquid formulations, tablets, capsules, and gelcaps. In some embodiments, the unit dose compositions are solid dosage forms, such as tablets and capsules. In some embodiments, the unit dose compositions are tablets.

Pharmaceutically acceptable excipients are excipients generally recognized as safe such as lactose, microcrystalline cellulose, starch, calcium carbonate, magnesium stearate, stearic acid, talc, colloidal silicon dioxide, mannitol, croscarmellose sodium, hydroxypropyl cellulose. In some embodiments, the pharmaceutical unit dose composition disclosed herein comprises a diluent, a disintegrant, a binder, and a lubricant. See generally, Remington's Pharmaceutical Sciences, 20th ed., Mack Publishing, Easton PA (2000), which is incorporated herein by reference in its entirety.

In one embodiment, the pharmaceutical unit dose composition disclosed herein comprises mannitol, microcrystalline cellulose, croscarmellose sodium, hydroxypropyl cellulose, and magnesium stearate.

Oral dosage forms can be prepared by standard pharmaceutical manufacturing techniques. Such techniques include, for example, wet granulation, wet milling, fluid bed drying, dry milling, lubrication, tableting, and aqueous film coating.

In some embodiments, the pharmaceutical unit dose compositions of the present disclosure comprise from about 1 mg to about 1000 mg of vibegron.

In some embodiments, the pharmaceutical unit dose compositions of the present disclosure comprise from about 2 mg to about 1000 mg, from about 3 mg to about 1000 mg, from about 4 mg to about 1000 mg, from about 5 mg to about 1000 mg, from about 10 mg to about 1000 mg, from about 15 mg to about 1000 mg, from about 20 mg to about 1000 mg, from about 25 mg to about 1000 mg, from about 30 mg to about 1000 mg, from about 40 mg to about 1000 mg, from about 50 mg to about 1000 mg, from about 60 mg to about 1000 mg, from about 70 mg to about 1000 mg, from about 80 mg to about 1000 mg, from about 90 mg to about 1000 mg, from about 100 mg to about 1000 mg, from about 150 mg to about 1000 mg, from about 200 mg to about 1000 mg, from about 250 mg to about 1000 mg, from about 300 mg to about 1000 mg, from about 350 mg to about 1000 mg, from about 400 mg to about 1000 mg, from about 450 mg to about 1000 mg, from about 500 mg to about 1000 mg, from about 550 mg to about 1000 mg, from about 600 mg to about 1000 mg, from about 650 mg to about 1000 mg, from about 700 mg to about 1000 mg, from about 750 mg to about 1000 mg, from about 800 mg to about 1000 mg, from about 850 mg to about 1000 mg, from about 900 mg to about 1000 mg, or from about 950 mg to about 1000 mg of vibegron.

In some embodiments, the pharmaceutical unit dose compositions of the present disclosure comprise from about 1 mg to about 950 mg, from about 1 mg to about 900 mg, from about 1 mg to about 850 mg, from about 1 mg to about 800 mg, from about 1 mg to about 750 mg, from about 1 mg to about 700 mg, from about 1 mg to about 650 mg, from about 1 mg to about 600 mg, from about 1 mg to about 550 mg, from about 1 mg to about 500 mg, from about 1 mg to about 450 mg, from about 1 mg to about 400 mg, from about 1 mg to about 350 mg, from about 1 mg to about 300 mg, from about 1 mg to about 250 mg, from about 1 mg to about 200 mg, from about 1 mg to about 150 mg, from about 1 mg to about 100 mg, from about 1 mg to about 90 mg, from about 1 mg to about 80 mg, from about 1 mg to about 70 mg, from about 1 mg to about 60 mg, from about 1 mg to about 50 mg, from about 1 mg to about 40 mg, from about 1 mg to about 30 mg, from about 1 mg to about 25 mg, from about 1 mg to about 20 mg, from about 1 mg to about 15 mg, from about 1 mg to about 10 mg, from about 1 mg to about 5 mg, from about 1 mg to about 4 mg, from about 1 mg to about 3 mg, or from about 1 mg to about 2 mg of vibegron.

In some embodiments, the pharmaceutical unit dose compositions of the present disclosure comprise from 25 mg to 150 mg of vibegron.

In some embodiments, the pharmaceutical unit dose compositions of the present disclosure comprise from about 30 mg to about 145 mg, about 35 mg to about 140 mg, about 40 mg to about 135 mg, about 45 mg to about 130 mg, about 50 mg to about 125 mg, about 55 mg to about 120 mg, about 60 mg to about 115 mg, about 65 mg to about 110 mg, about 70 mg to about 105 mg, about 75 mg to about 100 mg, about 80 mg to about 95 mg, or about 85 mg to about 90 mg of vibegron.

In some embodiments, the pharmaceutical unit dose compositions of the present disclosure comprise from about 25 mg to about 145 mg, about 25 mg to about 140 mg, about 25 mg to about 135 mg, about 25 mg to about 130 mg, about 25 mg to about 125 mg, about 25 mg to about 120 mg, about 25 mg to about 115 mg, about 25 mg to about 110 mg, about 25 mg to about 105 mg, about 25 mg to about 100 mg, about 25 mg to about 95 mg, about 25 mg to about 90 mg, about 25 mg to about 85 mg, about 25 mg to about 80 mg, about 25 mg to about 75 mg, about 25 mg to about 70 mg, about 25 mg to about 65 mg, about 25 mg to about 60 mg, about 25 mg to about 55 mg, about 25 mg to about 50 mg, about 25 mg to about 45 mg, about 25 mg to about 40 mg, about 25 mg to about 35 mg, or about 25 mg to about 30 mg of vibegron.

In some embodiments, the pharmaceutical unit dose compositions of the present disclosure comprise from about 30 mg to about 150 mg, about 35 mg to about 150 mg, about 40 mg to about 150 mg, about 45 mg to about 150 mg, about 50 mg to about 150 mg, about 55 mg to about 150 mg, about 60 mg to about 150 mg, about 65 mg to about 150 mg, about 70 mg to about 150 mg, about 75 mg to about 150 mg, about 80 mg to about 150 mg, or about 85 mg to about 150 mg, about 90 mg to about 150 mg, about 95 mg to about 150 mg, about 100 mg to about 150 mg, about 105 mg to about 150 mg, about 110 mg to about 150 mg, about 115 mg to about 150 mg, about 120 mg to about 150 mg, about 125 mg to about 150 mg, about 130 mg to about 150 mg, about 135 mg to about 150 mg, about 140 mg to about 150 mg, or about 145 mg to about 150 mg of vibegron.

In some embodiments, the pharmaceutical unit dose compositions of the present disclosure comprise from about 55 mg to about 100 mg, from about 60 mg to about 100 mg, from about 65 mg to about 100 mg, from about 70 mg to about 100 mg, from about 75 mg to about 100 mg, from about 80 mg to about 100 mg, from about 85 mg to about 100 mg, from about 90 mg to about 100 mg, or from about 95 mg to about 100 mg of vibegron.

In some embodiments, the pharmaceutical unit dose compositions of the present disclosure comprise from about 50 mg to about 95 mg, from about 50 mg to about 90 mg, from about 50 mg to about 85 mg, from about 50 mg to about 80 mg, from about 50 mg to about 75 mg, from about 50 mg to about 70 mg, from about 50 mg to about 65 mg, from about 50 mg to about 60 mg, or from about 50 mg to about 55 mg of vibegron.

In some embodiments, the pharmaceutical unit dose compositions of the present disclosure comprise from about 60 mg to about 90 mg, from about 65 mg to about 85 mg, or from about 70 mg to about 80 mg of vibegron. In some embodiments, the pharmaceutical unit dose compositions of the present disclosure comprise from 50 mg to 100 mg, from 60 mg to 90 mg, from 65 mg to 85 mg, or from 70 mg to 80 mg of vibegron.

In some embodiments, the pharmaceutical unit dose compositions of the present disclosure comprise about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg of vibegron. In some embodiments, the pharmaceutical unit dose compositions of the present disclosure comprise about 50 mg of vibegron. In some embodiments, the pharmaceutical unit dose compositions of the present disclosure comprise 50 mg of vibegron. In some embodiments, the pharmaceutical unit dose compositions of the present disclosure comprise about 75 mg of vibegron. In some embodiments, the pharmaceutical unit dose compositions of the present disclosure comprise 75 mg of vibegron. In some embodiments, the pharmaceutical unit dose compositions of the present disclosure comprise about 100 mg of vibegron. In some embodiments, the pharmaceutical unit dose compositions of the present disclosure comprise 100 mg of vibegron.

### In-Vitro Assays

Vibegron was tested in several in vitro assays to determine its agonist potency at human β3-AR, its selectivity versus the other human β-AR subtypes, and its potency at β3-ARs from other species.

Vibegron activity was measured in a functional assay measuring increases in cellular adenylyl cyclase activity in Chinese hamster ovary (CHO) cells stably expressing the human β3-AR. The degree of activation relative to a proven full agonist (isoproterenol) was measured along with the compound EC50.

Vibegron is a potent and selective agonist of β3-AR, with an EC50 of 1.1 nM and 84% activation relative to isoproterenol. A small serum shift is observed in the presence of 40% human serum (EC50=1.7 nM, 102% activation), consistent with the low plasma protein binding (49% unbound in human) of this compound.

In addition, the selectivity of vibegron for β3-AR over β1- and β2-AR subtypes was determined by testing in CHO cells expressing either β1-AR or β2-AR. Vibegron is highly selective over β1-AR and β2-AR versus β3-AR, demonstrating >9000-fold selectivity for activation of β3-AR over β1-AR or β2-AR in cell based in vitro functional assays.

The IC50 of vibegron was determined in a standard competition binding assay using membranes prepared from cells expressing recombinant β1, β2 or β3-AR. Vibegron has a β3-AR IC50=193 nM (86 ng/mL) for competition of a non-specific β-AR radiolabeled antagonist 125I-CYP in a filter binding assay. The relative lack of binding affinity compared to the potent in vitro agonist activity of vibegron at the human β(3-AR is related to the relative ability of the compound to compete for uncoupled versus coupled receptors which would both be measured by the antagonist binding assay. In addition, the compound does not bind to either β1-AR or β2-AR as demonstrated in binding competition assays, confirming that the compound is neither an agonist nor an antagonist at these receptors.

### Absorption, Distribution, Metabolism, and Excretion

Vibegron reaches maximum plasma concentrations (Cmax) at approximately 1 to 3 hours after oral administration in healthy volunteers. Mean Cmax and AUC increase in a greater than dose-proportional manner up to 400 mg. Steady state concentrations were achieved within 7 days of once daily dosing of vibegron. The steady state AUC geometric mean accumulation ratios were ˜2 in young male subjects and ˜2.8 in elderly subjects (male and female). Vibegron exposures in young Japanese male subjects were modestly increased (<2-fold) following single-dose administration relative to exposures in non-Japanese young male subjects.

Administration of multiple oral doses of 150 mg vibegron with food in healthy middle-aged and elderly females resulted in mean AUCO-24 and Cmax values of ˜42% and 59% on Day 1 and ˜20% and 43% on Day 14 compared to the same dose in the fasted state.

In a two-part, open-label, single-dose study to investigate the pharmacokinetics of vibegron in patients with hepatic insufficiency the apparent volume of distribution (Vd/F) for vibegron was approximately 9120 L. Vibegron is bound (approximately 49%) to human plasma proteins.

Vibegron is eliminated by a variety of pathways including urinary excretion, biliary excretion, and hepatic metabolism. While CYP3A4 is the predominant CYP responsible for in vitro metabolism, metabolism appears to only play a minor role in the elimination of vibegron. In a mass balance study in healthy subjects, the majority of the recovered dose was eliminated as unchanged vibegron. The mean total recovery of radioactivity in the excreta was 79%, with approximately 59% and 20% of the dose recovered in feces and urine, respectively.

Vibegron does not inhibit CYP2D6, a common pathway for drug metabolism (Rutman, et al., J. of Urology, 2019, v201, No. 4S, e231). In some embodiments, the subject is concomitantly taking or has taken a drug metabolized by CYP2D6.

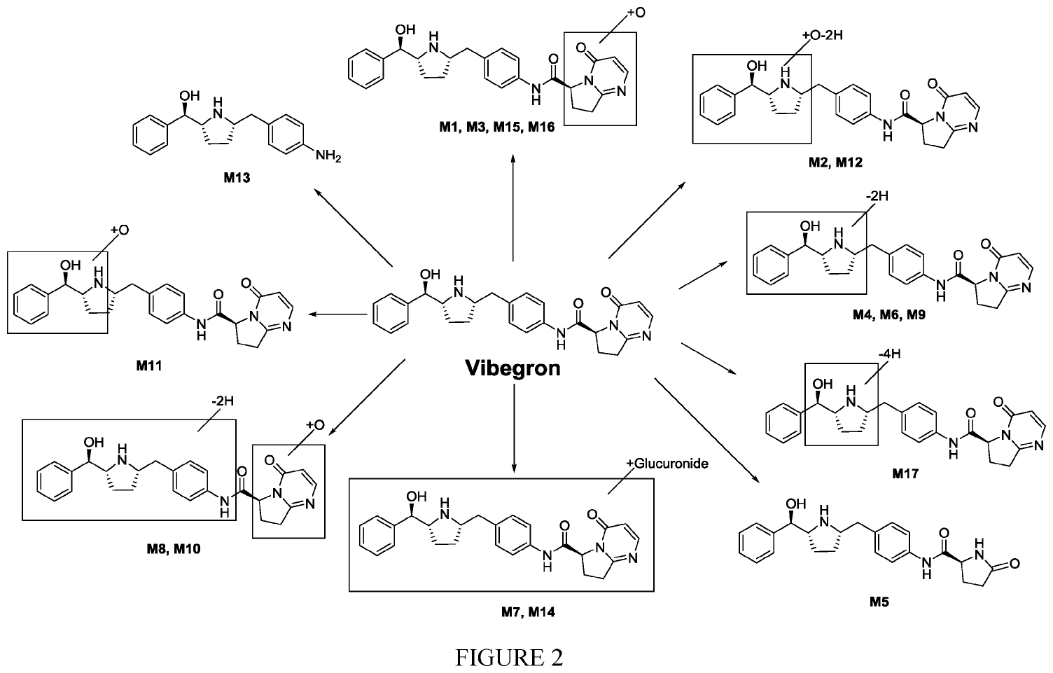

It was found that most of the vibegron dose was eliminated as the unchanged parent drug. Seven minor metabolites were detected in urine and feces, six of which (M1, M3, M4, M6, M11, and M17) were oxidative metabolites (see FIG. 2). The metabolite M7 is an O-glucuronide conjugate of vibegron. The concentration of [14C]vibegron derived radioactivity in plasma had an average Cmax of 0.3 μM and a Tmax of 2.5 hr. The radioactive profiles of plasma samples at 2 and 4 hr indicated that ˜78% and ˜73% of the plasma radioactivity, respectively, was accounted for by the unchanged vibegron, and the O-glucuronide (M7) was the predominant circulating metabolite (˜12-14% of the total circulating drug-related material). Two additional minor oxidative metabolites M4 (4-6%) and M17 (6-7%) were also detected in human plasma. The radioactivity in plasma samples at other time points beyond 4 h post dosing was too low to be profiled. The accumulation potential of circulating metabolites in plasma was not estimated due to insufficient data from later time points to enable estimation of half-life.

Vibegron has a terminal t1/2 of 59-94 hours in young and elderly subjects. At steady state, the average renal clearance (CLR) in young males ranged from 150 to 187 mL/min across all dose levels, while CLR in elderly subjects (male and female) was slightly less at 127 mL/min. There was a trend of increasing fraction of the dose excreted at steady state (fe0-24 hr, ss) with increasing dose, reflecting the increase in bioavailability as the dose increased. The fe0-24 hr, ss was similar in young males and elderly, ˜14% at 100 and 150 mg in young males and ˜17% at 100 mg in elderly subjects. The mean fe0-24 hr and CLR in young Japanese subjects were similar to what was observed in non-Japanese subjects.

### EXAMPLES

### Example 1

### Vibegron Tablet Formulation

The composition of vibegron tablets (50 mg, 75 mg, and 100 mg) is shown in Table 1.

TABLE 1
Vibegron Tablet Compositions

| | | Unit Strength | | |
|---|---|---|---|---|
| | | 50 mg | 75 mg | 100 mg |
| Components | Function | mg/tablet | mg/tablet | mg/tablet |
| Core Tablet | | | | |
| Vibegron | Active | 50.00 | 75.00 | 100.0 |
| Mannitol | Diluent | 20.75 | 31.125 | 41.50 |
| Microcrystalline Cellulose | Diluent | 20.75 | 31.125 | 41.50 |
| Croscarmellose Sodium | Disintegrant | 3.000 | 4.500 | 6.000 |
| Hydroxypropyl Cellulose | Binder | 4.500 | 6.75 | 9.000 |
| Magnesium Stearate | Lubricant | 1.000 | 1.500 | 2.000 |
| Purified Water¹ | Solvent | (35.00-45.00) | (52.5-67.5) | (70.00-90.00) |
| Total Core Weight | | 100.0 | 150.0 | 200.00 |
| Film Coating Suspension | | | | |
| Purified Water¹ | Solvent | (45.00) | (67.50 | (90.00) |
| OPADRY II Green | Colorant | 5.000 | 7.500 | 10.00 |
| (39K110004) | | | | |
| Total | | 105.0 | 157.5 | 210.0 |

¹Removed during processing

### Example 2

### Pharmacokinetic Data

### 2.1 Single-Dose Pharmacokinetics

Single-dose pharmacokinetics of vibegron were examined in two double-blind, randomized, placebo-controlled, single rising oral dose Phase 1 studies. All subjects were healthy adults. A summary of the results is presented in Table 2. Following single oral vibegron doses ranging from 2 to 600 mg, the average tₘₐₓ occurred between 0.8 and 3 hours after dosing. Terminal elimination t_{1/2} averaged 43 to 75 hours for all doses from 10 to 600 mg in healthy young male subjects. Systemic exposures were greater than dose proportional up to 600 mg.

Vibegron exposures in Japanese young males were modestly increased relative to exposures in non-Japanese young males. Geometric mean ratios (GMRs; Japanese/non-Japanese) for vibegron AUC_{0-inf} and corresponding 90% CIs decreased with increasing dose, from 1.75 (1.38, 2.23) at 10 mg to 1.17 (0.99, 1.40) at 300 mg. The GMR (Japanese/non-Japanese) and 90% CI for vibegron Cₘₐₓ did not appear to be influenced by dose and was 1.75 (1.35, 2.26) pooled across all doses. Median Tₘₐₓ values (1 to 3 hours) and harmonic mean apparent terminal t_{i!2} estimates (58 to 71 hours) in the Japanese subjects were similar to those in the non-Japanese subjects. Similar to non-Japanese subjects, AUC_{0-inf} and Cₘₐₓ in the Japanese subjects appeared to increase in a greater than dose proportional manner up to 300 mg.

Single-dose pharmacokinetics of 50 mg vibegron in non-Japanese elderly male and female subjects are also presented in Table 2. In elderly male and female subjects, mean AUC_{0-inf} and Cₘₐₓ following administration of 50 mg vibegron were ˜70% and 60% higher, respectively, relative to corresponding values following 50 mg in young males. Tₘₐₓ was similar to that observed in young males (median Tₘₐₓ=1.0 hr), while the apparent terminal t_{1/2} was slightly longer in elderly relative to young (harmonic mean t_{1/2}=92 vs. 52 hr). Vibegron exposures in elderly females were somewhat higher than in elderly males.

TABLE 2
Summary of Selected Single Dose Plasma Vibegron Pharmacokinetic Parameters

| Dose | | AUC_{0-inf} | AUC₀₋₂₄ | Cₘₐₓ | Tₘₐₓ^{b} | t_{1/2}^{c} |
|---|---|---|---|---|---|---|
| (mg)^{a} | N | (ng · h/mL) | (ng · h/mL) | (ng/mL) | (hr) | (hr) |
| 2 | 3^{d} | [symbol] | 0.80 ± 0.33 | 0.28 ± 0.02 | 3.0 (1.0-3.0)^{c} | [symbol] |
| 5 | 6 | [symbol] | 8.31 ± 4.80 | 0.79 ± 0.30 | 1.0 (0.5-6.0) | [symbol] |
| 10 | 6 | 70.7 ± 34.9 | 30.0 ± 12.6 | 4.76 ± 4.58 | 2.5 (1.0-6.0) | 43.2 ± 13.0 |
| 10 | 6 | 98.7 ± 27.3 | 31.0 ± 9.33 | 3.34 ± 1.97 | 1.0 (1.0-4.0) | 57.6 ± 39.0 |
| (Japanese) | | | | | | |
| 20 | 6 | 121 ± 48.9 | 40.0 ± 21.1 | 5.25 ± 4.25 | 0.8 (0.5-6.0) | 64.2 ± 12.6 |
| 50 | 6 | 551 ± 262 | 219 ± 123 | 31.7 ± 35.0 | 2.0 (0.5-6.0) | 52.0 ± 7.8 |
| 50 | 6 | 885 ± 241 | 385 ± 136 | 62.2 ± 20.4 | 3.0 (0.5-3.0) | 64.4 ± 8.7 |
| (Japanese) | | | | | | |
| 50 | 12 | 951 ± 300 | 314 ± 119 | 50.2 ± 23.6 | 1.0 (0.5-3.0) | 92.1 ± 15.9 |
| (Elderly Male | | | | | | |
| and Female) | | | | | | |
| 100 | 6 | 1890 ± 698 | 845 ± 401 | 142 ± 108 | 2.0 (1.0-4.0) | 72.8 ± 10.8 |
| 100 | 6 | 1770 ± 418 | 920 ± 300 | 190 ± 123 | 2.5 (0.5-4.0) | 57.6 ± 12.0 |
| (Japanese) | | | | | | |
| 150 | 6 | 2270 ± 911 | 1050 ± 551 | 195 ± 185 | 1.0 (1.0-6.0) | 60.5 ± 10.5 |
| 200 | 18 | 3630 ± 1110 | 1740 ± 748 | 274 ± 138 | 1.0 (1.0-4.0) | 75.3 ± 9.1 |
| 200 | 6 | 5200 ± 791 | 3090 ± 569 | 516 ± 200 | 2.0 (0.5-4.0) | 58.4 ± 9.0 |
| (Japanese) | | | | | | |
| 300 | 6 | 7380 ± 1410 | 4427 ± 996 | 618 ± 231 | 2.5 (2.0-3.0) | 63.4 ± 3.0 |
| 300 | 6 | 6270 ± 1570 | 4050 ± 1240 | 733 ± 210 | 2.0 (1.0-4.0) | 59.7 ± 9.2 |
| (Japanese) | | | | | | |
| 450 | 6 | 9157 ± 1850 | 5510 ± 1440 | 645 ± 165 | 3.0 (0.5-6.0) | 60.0 ± 9.4 |
| 600 | 5 | 15500 ± 3450 | 10900 ± 2770 | 1330 ± 529 | 3.0 (2.0-6.0) | 60.5 ± 5.2 |

Concentration data converted from molar to ng/mL (molecular weight of vibegron = 444.5)
^{a}Dosed in healthy young males unless otherwise indicated
^{b}Median (minimum-maximum)
^{c}Harmonic mean ± Pseudo SD
^{d}Only 3 of 6 subjects had any concentrations above the limit of quantitation at the 2 mg dose. Summary statistics for Cₘₐₓ, Tₘₐₓ and AUC₀₋₂₄ are based only on data from these subjects.
^{e}The duration of sampling was too short for 2 and 5 mg, precluding an accurate determination of the apparent terminal t_{1/2} and AUC_{0-inf}

### 2.2 Multiple-Dose Pharmacokinetics

The multiple-dose pharmacokinetics of vibegron were examined in healthy non-Japanese young male subjects, middle-aged male and female subjects, and elderly male and female subjects, and in healthy Japanese young male subjects, and elderly male and female subjects in two randomized, double-blind, placebo-controlled, multiple rising dose Phase 1 studies. Non-Japanese subjects received multiple doses ranging from 25 to 400 mg for 7 to 28 days, whereas Japanese subjects received multiple doses of 50 to 200 mg for 14 days. Pharmacokinetic results after 14 days of dosing are summarized in Table 3.

On average, females tend to have 50% higher exposures (AUC) compared with males, regardless of age. Steady state AUC and Cmax values following QD doses of 100 mg vibegron in elderly subjects were about 1.7-fold and 1.3-fold higher, respectively, compared to young males.

The GM Cmax and AUC accumulation ratio were 1.78 and 1.84 for Japanese subjects at the 200 mg dose level. On average, steady state exposures in the Japanese young male subjects were ˜30% higher than those in the young male non-Japanese subjects; differences in exposure were statistically significant. The GMR (Japanese/non-Japanese) and corresponding 90% CI of vibegron AUC and Cmax pooled across doses were 1.27 (1.09, 1.48) and 1.33 (1.06, 1.67), respectively.

On average, steady state exposures on Day 14 in elderly male and female Japanese subjects were 35% higher than those in elderly male and female non-Japanese subjects; differences in exposure were statistically significant. Day 14 GMR (Japanese/non-Japanese) and corresponding 90% CI of vibegron AUC0-24 and Cmax for the elderly panel were 1.35 (1.09, 1.68) and 1.82 (1.32, 2.51), respectively.

TABLE 3
Summary of Selected Multiple Dose Plasma Vibegron Pharmacokinetic Parameters

| Dose | | AUC₀₋₂₄ | Cₘₐₓ | C_{trough} | Tₘₐₓ^{b} | t_{1/2}^{c} |
|---|---|---|---|---|---|---|
| (mg)^{a} | N | (ng · h/mL) | (ng/mL) | (ng/mL) | (hr) | (hr) |
| 25 | 6 | 164 ± 25.9 | 15.6 ± 6.93 | 5.07 ± 0.711 | 1.0 (0.5-2.0) | 94.0 ± 9.60 |
| 50 | 6 | 507 ± 176 | 41.5 ± 12.3 | 15.2 ± 5.07 | 2.5 (0.5-6.0) | 77.2 ± 8.9 |
| 50 | 5 | 613 ± 296 | 56.9 ± 34.2 | 16.5 ± 6.05 | 3.0 (0.5-3.0) | 69.4 ± 6.6 |
| (Japanese) | | | | | | |
| 100 | 6 | 1280 ± 529 | 169 ± 80.9 | 31.9 ± 11.5 | 1.0 (0.5-4.0) | 79.7 ± 11.5 |
| 100 | 6 | 1710 ± 542 | 180 ± 111 | 41.0 ± 11.0 | 2.0 (2.0-4.0) | 56.8 ± 19.2 |
| (Japanese) | | | | | | |
| 100 | 12 | 2230 ± 671 | 224 ± 92.0 | 54.2 ± 15.3 | 1.0 (0.5-6.0) | 88.4 ± 10.7 |
| (Elderly Male | | | | | | |
| and Female) | | | | | | |
| 100 | 12 | 2920 ± 693 | 393 ± 165 | 57.3 ± 12.2 | 1.5 (0.5-4.0) | 75.1 ± 3.9 |
| (Elderly | | | | | | |
| Japanese) | | | | | | |
| 150 | 6 | 2285 ± 1140 | 305 ± 215 | 54.2 ± 16.6 | 1.5 (0.5-4.0) | 79.2 ± 9.2 |
| 150 | 9 | 2170 ± 452 | 293 ± 67.1 | 46.2 ± 8.50 | 1.0 (0.5-3.0) | 72.7 ± 16.1^{d} |
| (Middle-Aged | | | | | | |
| Male) | | | | | | |
| 150 | 9 | 3180 ± 925 | 246 ± 139 | 62.7 ± 12.4 | 2.0 (1.0-4.0) | 83.1 ± 17.2 |
| (Middle-Aged | | | | | | |
| Female) | | | | | | |
| 200 | 6 | 3200 ± 1120 | 313 ± 168 | 61.8 ± 12.4 | 2.0 (1.0-3.0) | 64.7 ± 6.5 |
| 200 | 6 | 4370 ± 618 | 631 ± 154 | 62.2 ± 9.07 | 1.0 (0.5-6.0) | 59.5 ± 1.9 |
| (Japanese) | | | | | | |
| 300 | 18 | 6980 ± 1040 | 733 ± 164 | 128 ± 23.6 | 2.0 (2.0-3.0) | 61.7 ± 7.3 |
| 400 | 6 | 10500 ± 2140 | 1400 ± 257 | 189 ± 54.7 | 1.5 (1.0-3.0) | 58.9 ± 5.9 |

Concentration data converted from molar to ng/mL (molecular weight of vibegron = 444.5)
^{a}Dosed in healthy young males unless otherwise indicated
^{b}Median (minimum-maximum)
^{c}Harmonic mean ± Pseudo SD
^{d}t_{1/2} determined after 28 days of dosing

### 2.3 Bioavailability and Bioequivalence

Five Phase 1 studies were conducted using a capsule formulation of vibegron, while seven Phase 1 studies and one Phase 2b study used a tablet formulation. An open-label randomized, 2-period, crossover PK study in healthy male subjects age 18 to 45 years compared single-dose pharmacokinetics of the capsule (1×150 mg capsule) and tablet (3×50 mg tablets) formulations of vibegron.

The tablet formulation provided comparable exposures to the capsule formulation as demonstrated in Table 4. Tₘₐₓ and the apparent terminal t_{1/2} were also similar between the two formulations.

TABLE 4
A Summary of the Effect of Formulation on the Pharmacokinetics
of 150 mg Vibegron in Healthy Male Subjects

| | Geometric Least | | | |
|---|---|---|---|---|
| Pharmacokinetic | Squares Mean (95% CI) | | | |
| Parameter | Capsule^{a} | Tablet^{b} | GMR | 90% CI |
| AUC_{0-inf} | 2840 | 2660 | 0.94 | (0.87, 1.00) |
| (ng · hr/mL) | (2512, 3220) | (2350, 3010) | | |
| Cₘₐₓ (ng/mL) | 237 | 213 | 0.90 | (0.75, 1.08) |
| | (190, 295) | (172, 264) | | |

Concentration data converted from molar to ng/mL (molecular weight of vibegron = 444.5)
GMR = Geometric least-squares mean ratio of tablet to capsule
^{a}1 × 150 mg vibegron capsule
^{b}3 × 50 mg vibegron tablets

An open-label, single-dose, randomized, two-period, two-treatment, two-sequence, crossover Phase 1 study evaluated the relative bioequivalence of two types of tablets with slightly different composition: aqueous tablet (test) and non-aqueous tablet (reference).

TABLE 5
A Summary of the Effect of Formulation on the Pharmacokinetics
of 50 mg Vibegron in Healthy Male and Female Subjects

| | Geometric Least | | | |
|---|---|---|---|---|
| | Squares Mean (95% CI) | | | |
| Pharmacokinetic | Reference | Test | GMR | |
| Parameter | Tablet^{a} | Tablet^{b} | (%) | 90% CI |
| AUC_{0-inf} | 671 | 671 | 100.2 | (91.6, 109.5.) |
| (ng · hr/mL) | (529, 853) | (547, 827) | | |
| Cₘₐₓ (ng/mL) | 38.0 | 41.0 | 107.7 | (87.4, 132.7) |
| | (27.8, 52.1) | (30.0, 56.1) | | |

Concentration data converted from molar to ng/mL (molecular weight of vibegron = 444.5)
GMR = Geometric least-squares mean ratio of Phase 3 tablet to Phase 2 tablet
^{a}Non-aqueous tablet (PMF1)
^{b}Aqueous (PMFII)

### 2.4 Effect of Food on Oral Absorption

The effect of food on the single dose pharmacokinetics of vibegron 50 mg was evaluated in healthy non-Japanese and Japanese young males in two randomized, double-blind, placebo-controlled, rising single-dose Phase 1 studies, while the effect of food on multiple dose pharmacokinetics of vibegron 150 mg in middle-aged females was evaluated in a randomized, double-blind, placebo-controlled, multiple rising dose Phase 1 study. A summary of the pharmacokinetic results are listed in Table 6.

Administration of 50 mg vibegron with a high-fat meal in non-Japanese young males resulted in 46% and 67% reductions in AUC_{0-inf} and Cₘₐₓ, respectively, and a delay in Tₘₐₓ of ˜1 hour compared to administration in the fasted state. Administration of 50 mg vibegron with a standard Japanese breakfast to Japanese young males resulted in 37% and 52% reductions in AUC_{0-inf} and Cₘₐₓ, respectively, roughly similar to findings in non-Japanese male subjects administered the same dose with a high fat meal.

Administration of multiple oral doses of 150 mg vibegron with food in healthy middle-aged females resulted in 20% and 47% reductions in mean AUC₀₋₂₄ₕᵣ and Cₘₐₓ, respectively, on Day 14 compared to the same dose in the fasted state. Tₘₐₓ at steady state was delayed in the fed state compared to the fasted state (6.0 vs. 2.0 hr).

TABLE 6
Summary of Food Effect on Vibegron Pharmacokinetic Parameters following
Single and Multiple Dose Administration in the Fed and Fasted
State to Healthy Japanese and Non-Japanese Young Male Subjects,
and to Healthy Non-Japanese Middle-Aged Female Subjects

| | | Pharmacokinetic Parameters^{a} | | | |
|---|---|---|---|---|---|
| Single Dose (mg) | | AUC_{0-inf} | AUC₀₋₂₄ | Cₘₐₓ | Tₘₐₓ^{b} |
| in Young Males | N | (ng · h/mL) | (ng · h/mL) | (ng/mL) | (hr) |
| 50 | 6 | 316 ± 127 | 90.7 ± 22.9 | 7.6 ± 2.27 | 3.0 (2.0-6.0) |
| (Non-Japanese, fed) | | | | | |
| 50 | 6 | 551 ± 262 | 219 ± 123 | 31.7 ± 35.0 | 2.0 (0.5-6.0) |
| (Non-Japanese, fasted) | | | | | |
| 50 | 6 | 605 ± 222 | 226 ± 112 | 36.2 ± 33.3 | 1.5 (0.5-3.0) |
| (Japanese, fed) | | | | | |
| 50 | 5 | 885 ± 241 | 385 ± 136 | 62.2 ± 20.4 | 3.0 (0.5-3.0) |
| (Japanese, fasted) | | | | | |
| Multiple Dose (mg) | | Pharmacokinetic Parameters^{a,c} | | | |
| in Non-Japanese | | AUC₀₋₂₄ | Cₘₐₓ | C_{trough} | Tₘₐₓ^{b} |
| Middle-Aged Females | N | (ng · h/mL) | (ng/mL) | (ng/mL) | (hr) |
| 150 (Fed) | 6 | 2540 ± 334 | 185 ± 32.3 | 65.3 ± 7.87 | 6.0 (3.0-6.0) |
| 150 (Fasted) | 9 | 3180 ± 925 | 346 ± 139 | 62.7 ± 12.4 | 2.0 (1.0-4.0) |

Concentration data converted from molar to ng/mL (molecular weight of vibegron = 444.5)
^{a}Geometric mean (CV %)
^{b}Median (minimum-maximum)
^{c}PK parameters obtained at day 14 of vibegron dosing

### 2.5 Pharmacokinetics in the Target Disease Population

A randomized, double-blind, placebo- and active-controlled, parallel-group two-part Phase 2b study in patients with OAB measured sparse vibegron trough concentrations (C_{trough}) only; the mean (±SD) C_{trough} of vibegron 50 mg and 100 mg QD were 27.4 (±18.3) ng/mL and 73.6 (±65.5) ng/mL, respectively. Mean (±SD) C_{trough} of vibegron 50 mg in healthy young men was 15.2 (±5.07) ng/mL. Mean (±SD) C_{trough} values of vibegron 100 mg ranged from 31.9 (±11.5) in healthy young men to 54.2 (±15.3) in healthy elderly.

### Example 3

### Pharmacokinetics in Special Populations

### 3.1 Effect of Age

Vibegron exposures were evaluated in young (18 to 45 years), middle-aged (46 to 64 years) and elderly (65 to 85 years) males and females. Although exposures were similar in middle-aged males when compared to young males, plasma concentrations were higher in elderly compared to middle-aged and young subjects. After a single 50 mg dose, vibegron AUC_{0-inf} and Cₘₐₓ were 70% and 60% higher, respectively in elderly subjects compared with young subjects. Elimination t_{1/2} was longer in the elderly at 92 hours compared to 52 hours in young subjects in a randomized, double-blind, placebo-controlled, rising single-dose study. Steady state vibegron AUC₀₋₂₄ and Cₘₐₓ values were ˜1.7-fold and ˜1.3-fold greater, respectively, in the elderly compared with young males in a randomized, double-blind, placebo-controlled, multiple rising dose study. Furthermore, the steady state AUC geometric mean accumulation ratios were ˜2 in young males and ˜2.8 in the elderly. In elderly Japanese, AUC₀₋₂₄ and Cₘₐₓ were increased by ˜35% and 82%, respectively compared to elderly non-Japanese.

### 3.2 Effect of Gender

The effect of gender on steady-state vibegron exposures after 100 or 150 mg doses was evaluated in a randomized, double-blind, placebo-controlled, multiple rising dose study. Vibegron plasma concentrations were similar in middle-aged males when compared to young males; however, exposures were slightly higher in middle-aged females compared to middle-aged males (˜1.5-fold higher steady state AUC in middle-aged females), which was also observed when comparing exposures in elderly females to those in elderly males.

### 3.3 Effect of Renal Impairment

The pharmacokinetics of single dose vibegron 100 mg in 24 patients with impaired renal function (8 severe, 8 moderate, and 8 mild) were compared to 8 healthy control subjects in an open-label, single-dose PK study. A summary of the pharmacokinetic parameters and a statistical comparison between patients with varying degrees of renal impairment and their healthy matched subjects are presented in Table 7.

Vibegron AUC_{0-inf} in patients with mild (eGFR≥60 to <90 mL/min/1.73 m2), moderate (eGFR≥30 to <60 mL/min/1.73 m2), and severe (eGFR<30 mL/min/1.73 m2 but not on dialysis) renal impairment were 49%, 106%, and 83% higher, respectively, compared to healthy matched control subjects. Vibegron Cₘₐₓ in mild, moderate, and severe renal impairment patients were 96%, 68%, and 42% higher, respectively, compared to healthy matched control subjects. In summary, increasing degree of renal impairment was associated with an increase in vibegron AUC_{0-inf} with no clear trend observed in Cₘₐₓ. Decreasing renal function was associated with lower clearance. The relationship between clearance and renal function was modeled using linear regression. Based on the slope from the regression, CL/F was found to increase ˜0.8% per one mL/min/1.73 m2 increase in eGFR. Based on this linear relationship, the CL/F ratio for mild, moderate, and severe populations relative to healthy subjects was predicted to be 0.81, 0.64, and 0.50, respectively. Corresponding predicted ratios for AUC were 1.24, 1.57, and 2.00. Modeling the relationship between CL/F and creatinine Clearance yielded similar results. Renal clearance (CLR) and the fraction of dose excreted in urine over the 48-hour collection interval (fe[urine]48hr) decreased with increasing degree of renal impairment. Patients with mild, moderate, and severe renal impairment had reduced CLR by 39%, 65%, and 82%, respectively, compared to healthy matched control subjects. The fe[urine]48 hr was comparable between mild renal impairment patients (8.5%) and healthy matched controlled subjects (7.9%) and was 5.5% and 2.1% in moderate and severe renal impairment patients, respectively.

TABLE 7
Summary of Vibegron 100 mg Pharmacokinetic Parameters in Patients with Severe,
Moderate and Mild Renal Impairment and Healthy Matched Control Subjects

| | | Geometric Least Squares Mean (95% CI) | | | |
|---|---|---|---|---|---|
| Pharmacokinetic | | Severe Renal | Moderate Renal | Mild Renal | Healthy Matched |
| Parameter | N | Impairment | Impairment | Impairment | Control Subjects |
| AUC_{0-inf} | 8 | 2820 | 3170 | 2290 | 1540 |
| (ng · hr/mL) | | (2200, 3610) | (2500, 4030) | (1800, 2920) | (1180, 2010) |
| Cₘₐₓ (ng/mL) | 8 | 152 | 180 | 210 | 107 |
| | | (103, 225) | (123-262) | (144, 308) | (70.8, 162) |
| CL/F (L/hr) | 8 | 35.5 | 31.5 | 43.6 | 64.9 |
| | | (27.68, 45.53) | (24.80, 40.06) | (34.23, 55.61) | (49.87, 84.47) |
| Tₘₐₓ^{a} (hr) | 8 | 0.5 | 1.3 | 1.0 | 1.5 |
| | | (0.5-4.0) | (0.5-3.0) | (0.5-3.0) | (0.5-4.0) |
| Apparent terminal | 8 | 131 | 108 | 96.2 | 98.8 |
| t_{1/2} ^{b} (hr) | | (10.0) | (21.0) | (11.5) | (13.9) |
| CL_{R} ^{b} (L/hr) | 8 | 1.9 | 3.6 | 6.3 | 10.4 |
| | | (30.9) | (34.5) | (31.1)^{c} | (20.2) |
| Fe[urine]48 hr ^{b} (%) | 8 | 2.1 | 5.5 | 8.5 | 7.9 |
| | | (57.6) | (53.2) | (43.9)^{c} | (43.0) |

| | GMR (90% CI) | | |
|---|---|---|---|
| Comparison | AUC_{0-inf} | Cₘₐₓ | CL/F |
| Patients with Severe Renal Impairment/ | 1.83 | 1.42 | 0.55 |
| Healthy Matched Control Subject | (1.36, 2.46) | (0.89, 2.27) | (0.41, 0.74) |
| Patients with Moderate Renal Impairment/ | 2.06 | 1.68 | 0.49 |
| Healthy Matched Control Subject | (1.55, 2.74) | (1.07, 2.63) | (0.36, 0.65) |
| Patients with Mild Renal Impairment/ | 1.49 | 1.96 | 0.67 |
| Healthy Matched Control Subject | (1.11, 2.00) | (1.23, 3.13) | (0.50, 0.90) |

Concentration data converted from molar to ng/mL (molecular weight of vibegron = 444.5)
CI = confidence interval;
GMR = Geometric least-squares mean ratio between treatment populations
^{a}Median (minimum-maximum)
^{b} Geometric mean (percent geometric coefficient of variation)
^{c}N = 7

### 3.4 Effect of Hepatic Impairment

The pharmacokinetics of a single dose of vibegron 100 mg were evaluated in 8 patients with moderate hepatic impairment (Child-Pugh Score of 7 to 9) and 8 healthy subjects matched for age, gender and BMI in a two-part, open-label, single-dose Phase 1 study. A statistical comparison of vibegron pharmacokinetic parameters is presented in Table 8. The AUC_{0-inf} and Cₘₐₓ GMRs (90% CI) for moderate hepatic impaired patients and healthy control subjects were 1.27 (0.96, 1.67) and 1.35 (0.88, 2.06), respectively suggesting that moderate hepatic impairment did not have a clinically important effect on the exposure of vibegron.

TABLE 8
Summary of Vibegron 100 mg Pharmacokinetic Parameters
in Patients with Moderate Hepatic Impairment
and Healthy Matched Control Subjects

| | | Geometric Least | | | |
|---|---|---|---|---|---|
| | | Squares Mean (95% CI) | | | |
| | | | Healthy | | |
| | | Moderate | Matched | | |
| Pharmacokinetic | | Hepatic | Control | | |
| Parameter | N | Impairment | Subjects | GMR | 90% CI |
| AUC_{0-inf} | 8 | 1820 | 1440 | 1.27 | (0.96, 1.67) |
| (ng · hr/mL) | | (1440, 2300) | (1140, 1810) | | |
| Cₘₐₓ (ng/mL) | 8 | 168 | 125 | 1.35 | (0.88, 2.06) |
| | | (118, 240) | (87.6, 178) | | |
| Tₘₐₓ^{a} (hr) | 8 | 1.0 | 1.5 | | |
| | | (0.5-3.0) | (0.5-4.0) | | |
| Apparent terminal | 8 | 94.5 | 92.5 | | |
| t_{1/2}^{b} (hr) | | (8.88%) | (9.37%) | | |
| CL/F^{b} (L/hr) | 8 | 56.0 | 68.3 | | |
| | | (31.2%) | (36.0%) | | |
| Vz/F^{b} (L) | 8 | 7640 | 9120 | | |
| | | (33.3%) | (30.7%) | | |

Concentration data converted from molar to ng/mL (molecular weight of vibegron = 444.5)
CI = confidence interval;
GMR = Geometric least-squares mean ratio between treatment populations
^{a}Median (minimum-maximum)
^{b}Geometric mean (percent geometric coefficient of variation)

### 3.5 Drug Interaction Studies

Four drug interaction studies evaluating vibegron in combination with six compounds were conducted. Table 9 summarizes the effect of ketoconazole, diltiazem or tolterodine on the pharmacokinetics of vibegron. Table 10 summarizes the effect of vibegron on the pharmacokinetics of digoxin, ethinyl estradiol, levonorgestrel or tolterodine.

Multiple doses of the strong CYP3A4/P-gp inhibitor, ketoconazole 200 mg and the moderate CYP3A4/P-gp inhibitor, diltiazem 240 mg were evaluated in combination with a single dose of vibegron 100 mg. GM vibegron AUC_{0-inf} and Cₘₐₓ increased 2.08-fold and 2.22 fold, respectively in the presence of multiple doses of 200 mg ketoconazole. GM vibegron AUC_{0-inf} and Cₘₐₓ increased 63% and 68%, respectively in the presence of multiple doses of 240 mg or 180 mg diltiazem. The GM t^{1/2} was 75, 75.4, and 80.2 hours, respectively when vibegron was dosed alone, with diltiazem or with ketoconazole, respectively. This lack of increase of vibegron t_{1/2} in the presence of ketoconazole or diltiazem suggests that the interaction occurred primarily in the absorption phase. However, these interactions are not expected to be clinically significant. Tolterodine ER 4 mg had no effect on the pharmacokinetics of vibegron.

Multiple doses of vibegron were evaluated in combination with the p-gp substrate, digoxin. The 90% CI for the AUC_{0-inf} GMR of digoxin when co-administered with vibegron was contained within the 80-125% bioequivalence range suggesting that vibegron does not influence digoxin pharmacokinetics to a clinically significant degree. The pharmacokinetics of ethinyl estradiol (EE) and levonorgestrel (LNG), two common components of oral contraceptives were not altered by multiple doses of vibegron. The 90% CI for the GMR (EE/LNG+vibegron to EE/LNG alone) for the AUC and Cₘₐₓ of EE were contained within 0.8 and 1.25. Although, LNG AUC and Cₘₐₓ increased 18 to 21% in the presence of multiple doses of vibegron, these increases were not considered to be clinically significant. No clinically meaningful pharmacokinetic interaction occurs when vibegron 100 mg or 150 mg is co-administered with tolterodine ER 4 mg.

TABLE 9
Change in Pharmacokinetic Parameters of Vibegron in
the Presence of Co-Administered Medication (Conmed)

| | | | | | | | Ratio (with/without | |
|---|---|---|---|---|---|---|---|---|
| | | | | | | | conmed) of Vibegron | |
| | | | | | | | Pharmacokinetic | |
| | Dose of | Dose of | | | Geometric Mean (95% CI) | | Parameters; | |
| | Conmed | Vibegron | | | Vibegron | Conmed + | No Effect = 1.00 | |
| Conmed | (mg) | (mg) | n | | alone | Vibegron | GMR | (90% CI) |
| Ketoconazole | 200 mg | 100 mg | 10 | AUC | 1370 | 2850 | 2.08 | (1.66, 2.61) |
| | every 12 | single | | | (788, 2380) | (2100, 3870) | | |
| | hours | dose | | Cₘₐₓ | 113 | 251 | 2.22 | (1.50, 3.28) |
| | | | | | (53.1, 241) | (167, 379) | | |
| Diltiazem ER | 240 mg | 100 mg | 12 | AUC | 1330 | 2170 | 1.63 | (1.44, 1.85) |
| | QD | single | | | (1130, 1570) | (1990, 2480) | | |
| | | dose | | Cₘₐₓ | 99.8 | 167 | 1.68 | (1.41, 1.99) |
| | | | | | (73.8, 135) | (129-217) | | |
| Tolterodine | 4 mg | 100 mg | 24 | AUC | 1662 | 1791^{a} | 1.08 | (0.94, 1.23) |
| ER | QD | QD | | | (1382, 2000) | (1533, 2094) | | |
| | | | | Cₘₐₓ | 158 | 163^{a} | 1.03 | (0.74, 1.43) |
| | | | | | (111, 224) | (127, 209) | | |
| | | 150 mg | 23 | AUC | 2783 | 3102^{a} | 1.12 | (0.98, 1.27) |
| | | QD | | | (2409, 3218) | (2787, 3463) | | |
| | | | | Cₘₐₓ | 269 | 304^{a} | 1.13 | (0.90, 1.42) |
| | | | | | (210, 344) | (260, 357) | | |

^{a}N = 12
Concentration data converted from molar to ng/mL (molecular weight of vibegron = 444.5)

TABLE 10
Drug Interactions: Change in Pharmacokinetic Parameters of
Co-Administered Drug (Conmed) in the Presence of Vibegron

| | | | | | | | Ratio (with/without | |
|---|---|---|---|---|---|---|---|---|
| | | | | | | | Vibegron) of Conmed | |
| | | | | | Geometric Mean (95% CI) of | | Pharmacokinetic | |
| | Dose of | Dose of | | | Conmed | | Parameters; No | |
| | Conmed | Vibegron | | | Conmed | Conmed + | Effect = 1.00 | |
| Conmed | (mg) | (mg) | n | | alone | Vibegron | GMR | (90% CI) |
| Digoxin | 0.25 mg | 100 mg | 18 | AUC | 16600 | 1840^{a} | 1.11 | (1.03, 1.19) |
| | single | QD | | | (14600, 19200) | (16200, 21000) | | |
| | dose | | | Cₘₐₓ | 1160 | 1410 | 1.21 | (1.09, 1.35) |
| | | | | | (965, 1400) | (1170, 1700) | | |
| Oral | 0.03 mg | 100 mg | 18 | AUC | 810 | 838 | 1.04 | (1.00, 1.07) |
| Contraceptive | EE single | QD | | | (713, 920) | (734, 958) | | |
| | dose | | | Cₘₐₓ | 71.9 | 68.8 | 0.96 | (0.90, 1.02) |
| | | | | | (62.3, 82.9) | (60.5, 78.3) | | |
| | 0.15 mg | | | AUC | 31000 | 37600 | 1.21 | (1.13, 1.30) |
| | LNG single | | | | (26800, 35900) | (32300, 43700) | | |
| | dose | | | Cₘₐₓ | 2070 | 2440 | 1.18 | (1.09, 1.27) |
| | | | | | (1770, 2420) | (2100, 2840) | | |
| Tolterodine | 4 mg | 100 mg | 12 | AUC | 28.37 | 30.66 | 1.08 | (0.97, 1.21) |
| ER | QD | QD | | | (15.03, 53.56) | (16.24, 57.89) | | |
| | | | | Cₘₐₓ | 2.28 | 2.57 | 1.12 | (1.00, 1.26) |
| | | | | | (1.32, 3.96) | (1.48, 4.45) | | |
| | | 150 mg | | AUC | 13.25^{a} | 10.80 | 1.23 | (1.11, 1.35) |
| | | QD | | | (7.39, 23.76) | (6.02, 19.38) | | |
| | | | | Cₘₐₓ | 1.26^{a} | 0.92 | 1.37 | (1.20, 1.57) |
| | | | | | (0.66, 2.39) | (0.48, 1.75) | | |

GMR = Geometric Means Ratio;
CI = confidence interval;
EE = ethinyl estradiol;
LNG = levonorgestrel
Concentration data converted from molar to ng/mL (molecular weight of vibegron = 444.5)
^{a}N = 17
b. N = 11

### 3.6 Effect on QT Interval Prolongation

The effect of vibegron on QTc interval was evaluated in a single oral dose study. Fifty-two healthy subjects received a single dose of 400 mg vibegron, a single dose of vibegron 200 mg, a single dose of moxifloxacin 400 mg and a single dose of placebo to match vibegron.

The 400 mg dose of vibegron resulted in a maximum LS mean difference (90%

CI) from placebo in QTcF of 4.60 (2.71, 6.48) msec at 1 hour post dose. A similar result was noted in QTcF after the 200 mg single dose where the maximum LS mean difference (90% CI) from placebo was 4.98 (3.07, 6.88) msec at 1 hour post dose. The upper limits of the 90% CIs of all of the mean differences fell below the target of 10 msec. (Table 11). A statistically significant effect of moxifloxacin on QTcF was observed.

The GM (CV%) Cₘₐₓ and AUC_{0-23.5hr} achieved following a single 200 mg dose were 366 (50.4) ng/mL and 2270 (37.3) ng·h/mL respectively. Vibegron Cₘₐₓ was 1.63-fold the value obtained in elderly subjects receiving multiple doses of 100 mg in a double-blind, randomized, placebo-controlled, alternating (Panels A and B), multiple-period, single rising oral dose Phase 1 study, while the AUC was similar. The GM (CV%) Cₘₐₓ and AUC_{0-23.5hr} achieved following a single dose of 400 mg were 1020 (39.9) ng/mL and 6450 (34.0) ng·h/mL respectively. These Cₘₐₓ and AUC_{0-23.5hr} values are 4.55-fold and 2.89-fold the values obtained in elderly subjects receiving multiple doses of vibegron 100 mg.

Target PK exposures at both the 200 mg and 400 mg dose levels were achieved. The steady state Cₘₐₓ and AUC₀₋₂₄ₕᵣ values achieved in elderly female subjects at the highest clinical dose of 100 mg were 278 ng/mL and 2620 ng·h/mL, respectively.

TABLE 11
Statistical Comparison for QTcF Change From Baseline Difference From Placebo (Vibegron −
Placebo) by Treatment and Time Point Relative to the Administration of a Dose of 400 mg
Vibegron, a Dose of 200 mg of Vibegron, and a Single Dose of Placebo to Vibegron

| | | Single Dose of | | | Single Dose of | | | Single Dose of | | |
|---|---|---|---|---|---|---|---|---|---|---|
| | | 400 mg Vibegron (msec) | | | 200 mg Vibegron (msec) | | | Placebo to Vibegron (msec) | | |
| Hour | | N | LS Mean | 95% CI | N | LS Mean | 95% CI | N | LS Mean | 95% CI |
| 0.5 | hour | 52 | 2.37 | (0.66, 4.07) | 50 | 1.90 | (0.16, 3.63) | 50 | −1.00 | (−2.74, 0.73) |
| 1 | hour | 52 | 4.49 | (2.78, 6.19) | 50 | 4.87 | (3.13, 6.60) | 50 | −0.11 | (−1.84, 1.63) |
| 2 | hour | 52 | 0.73 | (−0.97, 2.43) | 50 | 2.06 | (0.32, 3.79) | 50 | −0.08 | (−1.81, 1.65) |
| 3 | hour | 52 | −0.30 | (−2.00, 1.41) | 50 | 1.14 | (−0.59, 2.88) | 50 | 0.74 | (−0.99, 2.47) |
| 4 | hour | 52 | −2.53 | (−4.23, −0.82) | 50 | −0.40 | (−2.14, 1.33) | 50 | 0.43 | (−1.30, 2.17) |
| 6 | hour | 52 | −8.33 | (−10.03, −6.62) | 50 | −6.89 | (−8.63, −5.16) | 50 | −5.63 | (−7.37, −3.90) |
| 8 | hour | 52 | −11.60 | (−13.30, −9.89) | 50 | −9.59 | (−11.33, −7.86) | 50 | −8.36 | (−10.09, −6.62) |
| 10 | hour | 52 | −10.29 | (−11.99, −8.58) | 50 | −8.82 | (−10.56, −7.09) | 50 | −6.15 | (−7.89, −4.42) |
| 12 | hour | 52 | −7.10 | (−8.80, −5.39) | 50 | −6.82 | (−8.56, −5.09) | 50 | −3.10 | (−4.83, −1.37) |
| 23.5 | hour | 52 | −2.87 | (−4.57, −1.17) | 50 | −2.15 | (−3.88, −0.41) | 50 | −2.53 | (−4.26, −0.79) |

| | | Difference From Single Dose of Placebo to 400 | | Difference From Single Dose of Placebo to 200 | |
|---|---|---|---|---|---|
| | | mg Dose of Vibegron (msec) | | Dose of Vibegron (msec) | |
| Hour | | LS Mean Difference | 90% CI ^{a} | LS Mean Difference | 90% CI ^{a} |
| 0.5 | hour | 3.37 | (1.49, 5.25) | 2.90 | (1.00, 4.80) |
| 1 | hour | 4.60 | (2.71, 6.48) | 4.98 | (3.07, 6.88) |
| 2 | hour | 0.81 | (−1.07, 2.69) | 2.14 | (0.23, 4.04) |
| 3 | hour | −1.04 | (−2.92, 0.85) | 0.40 | (−1.50, 2.30) |
| 4 | hour | −2.96 | (−4.84, −1.08) | −0.83 | (−2.73, 1.07) |
| 6 | hour | −2.70 | (−4.58, −0.81) | −1.26 | (−3.16, 0.64) |
| 8 | hour | −3.24 | (−5.12, −1.36) | −1.24 | (−3.14, 0.66) |
| 10 | hour | −4.14 | (−6.02, −2.25) | −2.67 | (−4.57, −0.77) |
| 12 | hour | −4.00 | (−5.88, −2.11) | −3.72 | (−5.63, −1.82) |
| 23.5 | hour | −0.34 | (−2.22, 1.54) | 0.38 | (−1.52, 2.28) |

Abbreviations: LS mean, least square means, CI, confidence interval
400 mg vibegron: Single dose of 400 mg vibegron (8 × 50 mg tablets).
200 mg vibegron: Single dose of 200 mg vibegron (4 × 50 mg tablets vibegron + 4 × vibegron matching placebo tablets) Placebo: Single Dose of vibegron matching placebo (8 × vibegron matching placebo tablets).
QTcF results at baseline (arithmetic mean): Placebo = 407.38, 400 mg vibegron = 407.64, 200 mg vibegron = 406.75, Moxifloxacin = 407.77
^{a} The two-sided 90% confidence intervals are equivalent to one-sided upper 95% confidence intervals.

### Example 4

### Safety Data

### 4.1 Phase I Safety Data

Safety data from 16 Phase 1 studies, which include 15 completed Phase 1 studies and 1 study that was terminated early (this study was terminated for reasons unrelated to efficacy or safety) was collected. In the Phase 1 program, a total of 466 subjects received at least one dose of vibegron; 238 subjects received single doses ranging from 2 to 600 mg and 238 subjects received multiple doses ranging from 25 to 400 mg for up to 28 days. Across the Phase 1 program, vibegron has been generally well tolerated. There were no treatment-emergent serious adverse events (SAEs) or deaths reported, and the majority of adverse events (AEs) were transient and mild or moderate in intensity.

In Phase 1 studies, there were isolated occurrences of orthostatic hypotension (decrease in systolic blood pressure >20 mmHg and/or decrease in diastolic blood pressure >10 mmHg), with or without symptoms (e.g., lightheadedness, dizziness, presyncope). The incidence of orthostatic AEs following co-administration of vibegron 100 mg or 150 mg and tolterodine ER 4 mg was similar to the incidence of these AEs following administration of vibegron or tolterodine alone. At doses up to 100 mg in Phase 1 multiple dose studies, AEs such as postural dizziness, dizziness, presyncope, or syncope have not exhibited a clear dose-response relationship. However, postural dizziness appeared to increase at doses of 100 mg and above and the incidence of the AE “orthostatic hypotension with symptoms” has tended to be higher at vibegron doses >200 mg. There were no occurrences of orthostatic AEs when vibegron 100 mg was co-administered to subjects with essential hypertension who were on a stable regimen of either metoprolol (a representative beta-blocker), or amlodipine (a representative vasodilator).

Review of preliminary Phase 1 safety data suggest no clinically meaningful changes in laboratory safety parameters (chemistry, hematology and urinalyses) or ECG parameters, including PR, QRS and QTc intervals. A thorough QT study has been completed, which found no clinically meaningful effect on QTc or blood pressure

### 4.2 Phase II Safety Data

Phase 2 safety data from a single Phase 2B study that has completed in which 933 subjects received at least one dose of vibegron was collected. Subjects received vibegron doses ranging from 3 to 100 mg for up to 8 weeks during the main study (alone or in combination with tolterodine). Of those completing the parent study, 605 subjects received doses of vibegron 50 mg (alone) or vibegron 100 mg (alone or in combination with tolterodine 4 mg) for up to 52 weeks during an extension study. A placebo group was included in the main study, and a group that received tolterodine monotherapy was included in the main study and in the extension. There were no deaths reported during the study. Vibegron was generally well tolerated. No meaningful differences in the overall incidence or severity of AEs or drug-related AEs were observed among the treatment groups compared to placebo.

Adverse events were reported in 607 (43.6%) of the 1393 allocated subjects in the main study. The proportion of subjects with one or more AEs in the vibegron 50 mg and vibegron 100 mg treatment groups was similar to placebo (see Table 14). A higher proportion of subjects reported one or more AEs in the vibegron 15 mg and vibegron 50 mg+tolterodine 4 mg treatment groups compared to placebo. The most frequently reported AEs were dry mouth, headache, urinary tract infections (UTI), and nasopharyngitis. The incidence of dry mouth was higher in groups that received tolterodine (alone or with vibegron) compared to the placebo or vibegron monotherapy groups.

There were 221 subjects with drug-related AEs, with the lowest incidence of drug-related AEs reported in the vibegron 100 mg treatment group. The proportion of subjects with drug-related AEs was similar in the vibegron monotherapy groups compared to placebo and only slightly higher in the concomitant treatment groups compared to placebo or either monotherapy. The proportion of subjects who discontinued due to a drug-related AE was low and similar across all treatment groups.

There were a total of 9 SAEs reported in 8 subjects and occurred across the treatment groups (2 placebo; 1 vibegron 3 mg; 1 vibegron 50 mg; 3 tolterodine 4 mg; 1 vibegron 50 mg+tolterodine 4 mg). The reported SAEs were atrial fibrillation, anaphylactic reaction, lung adenocarcinoma stage IV, chronic obstructive pulmonary disease, hypertension, overdose, foot fracture, and in one subject both gastroesophageal reflux disease and dizziness occurred after a pan endoscopic procedure that prolonged hospitalization. No specific AE term was reported in more than 1 subject. All SAEs were considered unrelated to study drug by the investigator.

During the 52-week extension, no meaningful differences in overall incidences of adverse events or serious adverse events were observed among the treatment groups.

Adverse events were reported in 531 (62.8%) of the 845 subjects. The proportion of subjects with one or more AEs was similar across all treatment groups. The most frequently reported adverse events were UTI, nasopharyngitis, upper respiratory tract infection, and dry mouth. The incidence of dry mouth was higher in the tolterodine ER 4 mg treatment group compared to the other treatment groups. The incidence of constipation was higher in the concomitant treatment group compared to the monotherapy treatment groups.

The proportion of subjects with drug-related AEs was slightly higher for tolterodine ER 4 mg and the concomitant dose arm compared to the vibegron 50 mg and 100 mg treatment arms. The proportion of subjects who discontinued due to an AE or a drug-related AE was higher for tolterodine ER 4 mg compared to the other treatment groups. There were total of 46 SAEs reported in 41 subjects during the extension. An overall higher incidence rate was reported in the tolterodine ER 4 mg and vibegron 50 mg treatment groups compared to the vibegron 100 mg treatment group. There was one drug-related SAE of ileus paralytic reported in the tolterodine ER 4 mg treatment group; the subject was discontinued due to this AE.

Table 12 below summarizes adverse events commonly seen in the vibegron Phase 2 program in patients with overactive bladder.

TABLE 12
Adverse Events in ≥2% Subjects in Phase 2 Study (First 12 weeks of Treatment)

| | | | | | | | | | | | | | | | Vibegron | | |
|---|---|---|---|---|---|---|---|---|---|---|---|---|---|---|---|---|---|
| | | | | | | | | | | | | | | | 50 mg + | | |
| | | | | | | | | | | | | | Vibegron | | Tolterodine | | |
| | | | | | | | | | | | Tolterodine | | 100 mg + | | ER 4 mg/ | | |
| | | | Vibegron | | Vibegron | | Vibegron | | Vibegron | | ER | | tolterodine | | Vibegron | | |
| | Placebo | | 3 mg | | 15 mg | | 50 mg | | 100 mg | | 4 mg | | ER 4 mg | | 50 mg | | Total |
| | N = 205 | | N = 144 | | N = 134 | | N = 148 | | N = 261 | | N = 257 | | N = 110 | | N = 134 | | N = 1,393 |
| | n (%) | | n (%) | | n (%) | | n (%) | | n (%) | | n (%) | | n (%) | | n (%) | | n (%) |
| ≥1 AE | 88 | (42.9) | 55 | (38.2) | 70 | (52.2) | 62 | (41.9) | 107 | (41.0) | 116 | (45.1) | 40 | (36.4) | 69 | (51.5) | |
| Serious AE | 2 | (1.0) | 1 | (0.7) | 0 | | 1 | (0.7) | 0 | | 3 | (1.2) | 0 | | 8 | (0.7) | |
| Drug-related AE | 30 | (14.6) | 21 | (14.6) | 23 | (17.2) | 23 | (15.5) | 31 | (11.9) | 42 | (16.3) | 21 | (19.1) | 30 | (14.6) | |
| Discontinuation | 5 | (2.4) | 3 | (2.1) | 4 | (3.0) | 2 | (1.4) | 6 | (2.3) | 4 | (1.6) | 2 | (1.8) | 3 | (2.2) | |
| due to AE | | | | | | | | | | | | | | | | | |

| | | | | | | | | | | | | | | |
|---|---|---|---|---|---|---|---|---|---|---|---|---|---|---|
| Discontinuation | 3 | (1.5) | 2 | (1.4) | 4 | (3.0) | 0 | 3 | (1.1) | 0 | 1 | (0.9) | 2 | (1.5) |
| due to | | | | | | | | | | | | | | |
| drug-related AE | | | | | | | | | | | | | | |

SOC/Preferred Term
Eye disorders

| | | | | | | | | | | | | | | | | | | |
|---|---|---|---|---|---|---|---|---|---|---|---|---|---|---|---|---|---|---|
| Dry eye | 10 | (4.9) | 2 | (1.4) | 4 | (3.0) | 2 | (1.4) | 4 | (1.5) | 10 | (3.9) | 3 | (2.7) | 2 | (1.5) | 18 | (1.3) |

Gastrointestinal disorders

| | | | | | | | | | | | | | | | | | | |
|---|---|---|---|---|---|---|---|---|---|---|---|---|---|---|---|---|---|---|
| Constipation | 5 | (2.4) | 5 | (3.5) | 6 | (4.5) | 6 | (4.1) | 2 | (0.8) | 5 | (1.9) | 4 | (3.6) | 6 | (4.5) | 39 | (2.8) |
| Diarrhea | 5 | (2.4) | 4 | (2.8) | 2 | (1.5) | 1 | (0.7) | 5 | (1.9) | 9 | (3.5) | 1 | (0.9) | 6 | (4.5) | 33 | (2.4) |
| Dry mouth | 6 | (2.9) | 5 | (3.5) | 6 | (4.5) | 7 | (4.7) | 4 | (1.5) | 22 | (8.6) | 13 | (11.8) | 11 | (8.2) | 74 | (5.3) |
| Nausea | 3 | (1.5) | 2 | (1.4) | 2 | (1.5) | 3 | (2.0) | 3 | (1.1) | 6 | (2.3) | 0 | (0.0) | 2 | (1.5) | 21 | (1.5) |

General disorders and administration site conditions

| | | | | | | | | | | | | | | | | | | |
|---|---|---|---|---|---|---|---|---|---|---|---|---|---|---|---|---|---|---|
| Fatigue | 1 | (0.5) | 4 | (2.8) | 6 | (4.5) | 5 | (3.4) | 2 | (0.8) | 6 | (2.3) | 2 | (1.8) | 2 | (1.5) | 28 | (2.0) |

Infections and infestations

| Nasopharyngitis | 14 | (6.8) | 3 | (2.1) | 7 | (5.2) | 8 | (5.4) | 10 | (3.8) | 4 | (1.6) | 2 | (1.8) | 3 | (2.2) | 51 | (3.7) |
|---|---|---|---|---|---|---|---|---|---|---|---|---|---|---|---|---|---|---|
| Sinusitis | 2 | (1.0) | 0 0 | | 2 | (1.5) | 1 | (0.7) | 0 0 | | 1 | (0.4) | 0 | (0.0) | 4 | (3.0) | 10 | (0.7) |
| Urinary tract | 7 | (3.4) | 5 | (3.5) | 5 | (3.7) | 8 | (5.4) | 8 | (3.1) | 12 | (4.7) | 5 | (4.5) | 7 | (5.2) | 57 | (4.1) |
| infection | | | | | | | | | | | | | | | | | | |

Injury, poisoning and procedural complications

| | | | | | | | | | | | | | | | | | | |
|---|---|---|---|---|---|---|---|---|---|---|---|---|---|---|---|---|---|---|
| Accidental | 2 | (1.0) | 3 | (2.1) | 6 | (4.5) | 4 | (2.7) | 11 | (4.2) | 6 | (2.3) | 1 | (0.9) | 2 | (1.5) | 35 | (2.5) |
| overdose | | | | | | | | | | | | | | | | | | |

Investigations

| | | | | | | | | | | | | | | | | | | |
|---|---|---|---|---|---|---|---|---|---|---|---|---|---|---|---|---|---|---|
| Alanine | 0 | (0.0) | 0 | (0.0) | 0 | (0.0) | 0 | (0.0) | 0 | (0.0) | 1 | (0.4) | 0 | (0.0) | 3 | (2.2) | 4 | (0.3) |
| aminotransferase | | | | | | | | | | | | | | | | | | |
| increased | | | | | | | | | | | | | | | | | | |
| Aspartate | 0 | (0.0) | 0 | (0.0) | 1 | (0.7) | 0 | (0.0) | 0 | (0.0) | 0 | (0.0) | 0 | (0.0) | 3 | (2.2) | 4 | (0.3) |
| aminotransferase | | | | | | | | | | | | | | | | | | |
| increased | | | | | | | | | | | | | | | | | | |

Musculoskeletal and connective tissue disorders

| Arthralgia | 2 | (1.0) | 0 0 | | 2 | (1.5) | 3 | (2.0) | 0 | (0.0) | 3 | (1.2) | 1 | (0.9) | 0 | (0.0) | 11 | (0.8) |
|---|---|---|---|---|---|---|---|---|---|---|---|---|---|---|---|---|---|---|
| Osteoarthritis | 1 | (0.5) | 2 | (1.4) | 1 | (0.7) | 4 | (2.7) | 1 | (0.4) | 0 | (0.0) | 0 | (0.0) | 0 | (0.0) | 9 | (0.6) |

| | | | | | | | | | | | | | | | | |
|---|---|---|---|---|---|---|---|---|---|---|---|---|---|---|---|---|
| Pain in extremity | 0 0 | 2 | (1.4) | 0 0 | 2 | (1.4) | 1 | (0.4) | 1 | (0.4) | 3 | (2.7) | 2 | (1.5) | 11 | (0.8) |

Nervous system disorders

| | | | | | | | | | | | | | | | | | | |
|---|---|---|---|---|---|---|---|---|---|---|---|---|---|---|---|---|---|---|
| Dizziness | 5 | (2.4) | 1 | (0.7) | 6 | (4.5) | 3 | (2.0) | 7 | (2.7) | 5 | (1.9) | 3 | (2.7) | 1 | (0.7) | 31 | (2.0) |
| Headache | 9 | (4.4) | 3 | (2.1) | 6 | (4.5) | 6 | (4.1) | 12 | (4.6) | 9 | (3.5) | 7 | (6.4) | 6 | (4.5) | 58 | (4.0) |

Renal and urinary disorders

| | | | | | | | | | | | | | | | | | | |
|---|---|---|---|---|---|---|---|---|---|---|---|---|---|---|---|---|---|---|
| Dysuria | 1 | (0.5) | 0 | (0.0) | 0 | (0.0) | 1 | (0.7) | 0 | (0.0) | 3 | (1.2) | 3 | (2.7) | 0 | (0.0) | 8 | (0.6) |

Serious adverse events observed during the first 12 weeks of treatment with vibegron monotherapy included lung adenocarcinoma stage IV (n=1) and chronic obstructive pulmonary disease (n=1); an SAE of overdose was reported in the vibegron-tolterodine combination arm. During the Phase 2 extension study, SAEs reported by 2 or more subjects receiving monotherapy included cerebrovascular accident (n=2) and osteoarthritis (n=2). The only SAE reported in the vibegron—tolterodine combination arm was borrelia infection. SAEs potentially related to a change in heart rate or blood pressure (at any time during treatment) included: loss of consciousness after 8 weeks of vibegron that did not recur on rechallenge (n=1), and in the tolterodine monotherapy arm atrial fibrillation (n=1) and dizziness (n=1). The frequency of injuries was numerically higher in the tolterodine arm than with vibegron (2.1%, n=5, vs. 0.9%, n=4). Given the low incidence and lack of a pattern for SAEs, no serious event is considered expected for vibegron.

Potential risks that may be associated with vibegron treatment, based on nonclinical data and data available for similar compounds, include orthostatic hypotension and increased exposure (˜2-fold) in patients taking concomitant strong P-gp inducers.

### 4.3 Cardiovascular Safety

The cardiovascular safety of vibegron has been evaluated in patients with OAB and healthy volunteers. In a randomized, placebo- and active comparator (tolterodine)-controlled, 2-part efficacy and safety study with 52-week extension, seven orthostatic related AEs (which included the adverse event terms of postural dizziness, presyncope, and orthostatic hypotension) occurred in 6 (0.4%) subjects. The events occurred in one subject each in the placebo group (0.5%), the vibegron 15 mg group (0.3%), and the vibegron 50 mg+tolterodine ER/vibegron 50 mg treatment group (0.8%), and in 3 subjects in the vibegron 100 mg group (1.1%). The events occurred at random times throughout the study and were judged by the investigator to be mild in severity. None led to discontinuation. The overall incidence of orthostatic symptoms was low.

Changes from baseline in BP and HR across treatment groups are shown in Table 13. For systolic blood pressure (SBP) and diastolic blood pressure (DBP), the mean changes at Week 1 and mean maximum changes over 8 weeks for 50 mg and 100 mg were comparable between placebo and vibegron, with differences of <1 mm Hg.

Categorical changes in SBP and DBP also were similar between placebo and vibegron, with a slight increase at 100 mg in percent of vibegron subjects with a change from baseline in DBP>15 mmHg (1.3% 100 mg vs 0.5% placebo). No dose-dependent pattern was detectable for HR, as the mean maximum changes over 8 weeks were comparable to placebo (<2 bpm). Small differences in the percent of subjects exceeding categorical heart rate and blood pressure thresholds for vibegron were similar to those in the tolterodine arm.

TABLE 13
Vital Sign Changes from Baseline for Vibegron and Tolterodine by Dose

HR Change from Baseline

| | | Mean (95% CI) | | Mean (95% CI) | ≥5 mm Hg | ≥10 bpm | ≥15 bpm |
|---|---|---|---|---|---|---|---|
| Treatment | n | (week 1) | n | Maximum | n/N (%) | n/N (%) | n/N (%) |
| Placebo | 186 | 0.12 (−1.04, 1.28) | 200 | 5.08 (4.02, 6.13) | 17/188 (9.0) | 1/188 (0.5) | 0 |
| 3 mg | 141 | 0.36 (−0.90, 1.62) | 143 | 5.57 (4.25, 6.90) | 16/140 (11.4) | 5/140 (3.6) | 1/140 (0.7) |
| 15 mg | 126 | 0.35 (−1.15, 1.85) | 134 | 6.56 (5.20, 7.92) | 17/132 (12.9) | 4/132 (3.0) | 1/132 (0.8) |
| 50 mg | 140 | 0.29 (−0.88, 1.46) | 146 | 5.49 (4.28, 6.69) | 12/144 (8.3) | 4/144 (2.8) | 1/144 (0.7) |
| 100 mg | 237 | 0.35 (−0.69, 1.38) | 257 | 6.12 (5.10, 7.15) | 28/237 (11.8) | 7/237 (3.0) | 1/237 (0.4) |
| Tolterodine 4 mg | 246 | 0.68 (−0.31, 1.67) | 257 | 5.66 (4.69, 6.63) | 29/242 (12.0) | 11/242 (4.5) | 4/242 (1.7) |

SBP Change from Baseline

| | | Mean (95% CI) | | Mean (95% CI) | ≥5 mm Hg | ≥10 mm Hg | ≥15 mm Hg |
|---|---|---|---|---|---|---|---|
| Treatment | n | (week 1) | n | Maximum | n/N (%) | n/N (%) | n/N (%) |
| Placebo | 186 | −0.21 (−1.85, 1.43) | 200 | 7.84 (6.27, 9.40) | 24/188 (12.8) | 10/188 (5.3) | 3/188 (1.6) |
| 3 mg | 141 | −0.35 (−2.34, 1.65) | 143 | 7.14 (5.18, 9.10) | 21/140 (15.0) | 10/140 (7.1) | 4/140 (2.9) |
| 15 mg | 126 | −0.34 (−2.46, 1.78) | 134 | 8.93 (7.18, 10.67) | 22/132 (16.7) | 9/132 (6.8) | 1/132 (0.8) |
| 50 mg | 140 | −0.79 (−2.65, 1.08) | 146 | 7.01 (5.31, 8.70) | 24/144 (16.7) | 14/144 (9.7) | 3/144 (2.1) |
| 100 mg | 237 | −0.77 (−2.22, 0.68) | 257 | 6.51 (5.09, 7.93) | 28/237 (11.8) | 10/237 (4.2) | 3/237 (1.3) |
| Tolterodine 4 mg | 246 | 0.04 (−1.36, 1.43) | 257 | 7.29 (6.01, 8.57) | 41/242 (16.9) | 19/242 (7.9) | 7/242 (2.9) |

DBP Change from Baseline

| | | Mean (95% CI) | | Mean (95% CI) | ≥5 mm Hg | ≥10 mmHg | ≥15 mm |
|---|---|---|---|---|---|---|---|
| Treatment | n | (week 1) | n | Max | n/N (%) | n/N (%) | n/N Hg (%) |
| Placebo | 186 | 0.11 (−0.94, 1.17) | 200 | 4.89 (3.89, 5.89) | 18/188 (9.6) | 6/188 (3.2) | 1/188 (0.5) |
| 3 mg | 141 | −0.37 (−1.69, 0.95) | 143 | 5.03 (3.92, 6.15) | 14/140 (10.0) | 3/140 (2.1) | 1/140 (0.7) |
| 15 mg | 126 | 0.03 (−1.52, 1.59) | 134 | 6.37 (5.20, 7.53) | 15/132 (11.4) | 3/132 (2.3) | 1/132 (0.8) |
| 50 mg | 140 | −0.70 (−2.07, 0.67) | 146 | 4.19 (3.10, 5.29) | 11/144 (7.6) | 5/144 (3.5) | 1/144 (0.7) |
| 100 mg | 237 | −0.69 (−1.72, 0.34) | 257 | 4.80 (3.88, 5.72) | 31/237 (13.1) | 8/237 (3.4) | 3/237 (1.3) |
| Tolterodine 4 mg | 246 | −0.12 (−1.10, 0.86) | 257 | 5.19 (4.26, 6.13) | 30/242 (12.4) | 13/242 (5.4) | 3/242 (1.2) |

Mean maximum is from week 1 to 8.
Counts based on 3 Consecutive Post-Baseline Visits.

More intensive assessments of heart rate and blood pressure were performed in healthy volunteers in several Phase 1 studies. A 6-part, double-blinded, randomized, placebo-controlled study to assess the safety, tolerability and multiple-dose PK of vibegron in healthy subjects that included specific analyses for heart rate. Doses ranged from 25 to 400 mg once daily for 7 to 28 days depending on the cohort. Least squares mean and 90% confidence intervals of maximum change from baseline in moving average of heart rate over 4 hours postdose (MA4 HR) are presented in Table 14. Effects on heart rate were dose dependent and the 100 mg dose demonstrated a <1 bpm difference from placebo.

TABLE 14
Maximum MA4 HR and Difference between
Vibegron and Placebo at Day 14

| Panel | Dose (mg) | N^{a} | _{Maximum}MA4 HR^{b} | | Difference from Placebo^{c} | |
|---|---|---|---|---|---|---|
| All | Placebo | 14 | 3.07 | (0.26, 5.88) | | |
| A | 25 | 5 | 1.47 | (−3.24, 6.17) | −1.60 | (−7.09, 3.88) |
| B | 50 | 6 | 1.50 | (−2.79, 5.79) | −1.57 | (−6.70, 3.56) |
| C | 100 | 6 | 3.28 | (−1.02, 7.57) | 0.21 | (−4.93, 5.34) |
| D | 150 | 6 | 3.17 | (−1.13, 7.46) | 0.10 | (−5.04, 5.23) |
| G | 200 | 5 | 5.67 | (0.96, 10.37) | 2.60 | (−2.89, 8.08) |
| H | 300 | 6 | 9.06 | (4.76, 13.35) | 5.98 | (0.85, 11.12) |
| I | 400 | 6 | 10.33 | (6.04, 14.63) | 7.26 | (2.13, 12.39) |

^{a}One subject each in panels A and G discontinued and had no available data at day 14
^{b}Least-square mean and corresponding 90% confidence interval
^{c}Difference of least squares (active − placebo) and corresponding 90% confidence interval calculated from the linear fixed effects model

Cardiovascular safety was also assessed in healthy volunteers in the thorough QT study following single doses of 200 and 400 mg, which approximate vibegron steady-state exposures at 100 mg and 200 mg, respectively. Mean maximum effects on blood pressure and RR interval were reduced with the lower dose as shown in Table 15. Using a log-log regression analysis from multiple-dose vibegron exposures (from three Phase 1 studies), the calculated mean±standard deviation Cₘₐₓ and AUC from a 75 mg dose were 120±74.7 ng/mL and 1140±476 ng·h/mL, respectively. These estimations represent a Cₘₐₓ and AUC that are approximately 3.3-fold and 2-fold lower, respectively, than the 200 mg single dose and 9.2-fold and 6-fold lower, respectively, than the 400 mg single dose.

TABLE 15
Single-Dose Pharmacokinetic Parameters and Mean Placebo-Corrected
Change from Baseline RR interval and Blood Pressure

| | | | Maximum Mean | Maximum Mean | Maximum Mean |
|---|---|---|---|---|---|
| | | | Placebo Corrected | Placebo Corrected | Placebo Corrected |
| | Mean ± SD | Mean ± SD | Change from Baseline | Change from Baseline | Change from Baseline |
| Dose | Cₘₐₓ | AUC | RR interval | Systolic BP | Diastolic BP |
| (mg) | (ng/mL) | (ng · h/mL) | (90% CI) (msec) | (90% CI) (mmHg) | (90% CI) (mmHg) |
| Vibegron | 1100 ± 436 | 6800 ± 2300 | −162.45 | 3.97 | 3.99 |
| 400 | | | (−184.24, −140.65) | (2.12, 5.81) | (2.62, 5.36) |
| Vibegron | 406 ± 180 | 2430 ± 974 | −84.36 | 2.20 | 2.42 |
| 200 | | | (−106.37, −62.35) | (0.34, 4.06) | (1.04, 3.81) |

### Example 5

### Dose Selection

### 5.1 Mitigating Side Effects

Vibegron demonstrates greater than a dose proportional increase in exposures.

Surprisingly, an increase in dose from 50 to 100 mg results in an approximate 3-fold increase in Cₘₐₓ, the PK parameter considered most closely associated with cardiovascular effects. In order to contextualize PK parameters of a 75 mg dose, dose-Cₘₐₓ and dose-AUC models were created using data from Phase 1 studies. Based on simulations, it was found that a vibegron dose of 75 mg avoids approximately 29% of the exposures observed with a 100 mg dose, subsequently reducing the upper range of exposures that would be achieved with a 100 mg dose. This reduction in outlier Cₘₐₓ values reduces the potential for clinically relevant cardiovascular effects.

In Phase 1 multiple dose studies, at doses up to 100 mg, adverse events such as postural dizziness, dizziness, presyncope, syncope did not exhibit a clear dose-response relationship. However, postural dizziness appeared to increase at doses ≥100 mg and the incidence of the adverse event “orthostatic hypotension with symptoms” was higher at vibegron doses greater than 150 mg. The risk of these dose-related adverse events can be disproportionally reduced by decreasing the dose from 100 mg to 75 mg, as a 25% reduction in dose produces an approximate 40% reduction in Cₘₐₓ (120 ng/mL with 75 mg vs. 206 ng/mL with 100 mg). Without wishing to be bound by theory, the greater than dose-proportional increase in bioavailability with increasing dose may be due to saturable P-glycoprotein (P-gp)-mediated efflux in the gut.

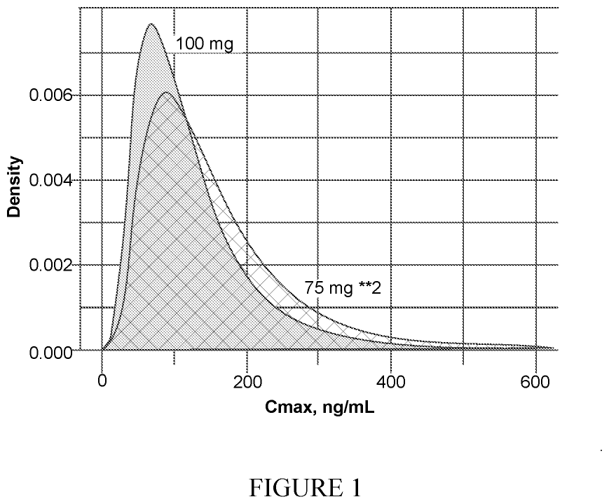

A lower exposure with the 75 mg dose compared to a 100 mg dose disproportionally reduces the risk of adverse events in special populations as well. Subjects with moderate renal impairment had a mean increase in AUC of 1.6-fold compared to subjects with normal renal function whereas subjects receiving a potent CYP3A/P-gp inhibitor had an approximate 2-fold higher exposure. Assuming a 2 fold increase in Cₘₐₓ of a 75 mg dose, the probability of these special populations achieving a vibegron Cₘₐₓ greater than those observed with 100 mg is 15% (see FIG. 1). Minimizing exposures of subjects who fall at the extremes is important for elderly and females who demonstrated approximately a 50-70% higher Cₘₐₓ than healthy young males.

### Example 6

### A Phase 2, Randomized, Double-Blind, Placebo-Controlled Study to Evaluate the Efficacy, Safety and Tolerability of Vibegron Administered Orally for 12 Weeks to Patients With Irritable Bowel Syndrome (IBS)

A Phase 2a study will evaluate the efficacy, safety, and tolerability of vibegron versus placebo for treating pain associated with irritable bowel syndrome that is associated with predominantly diarrheal (IBS-D) or that has mixed episodes of diarrhea and constipation (IBS-M). At least one vibegron dose is more effective than placebo in improving primary endpoints, i.e., Abdominal Pain Intensity (API) responder, defined as the decrease in weekly average of worst abdominal pain in the past 24 hours score of at least 30% compared with baseline weekly average. Based on previous studies with vibegron, treatment is expected to be safe and well tolerated in patients with IBS. Additional measures of outcome will include patient rating of treatment on a Global Impression Scale and IBS Quality of life responder rates.

The study design follows a 2-week double-blind placebo run-in period, after which female subjects will be randomized in a 1:1 fashion to receive either 75 mg vibegron or placebo for a 12-week double-blind treatment period, followed by a follow-up visit 3 weeks after the end of the double-blind treatment. The study visit schedules are Visit 1 (Screening), Visit 2 (run-in 2 weeks), Visit 3 (Baseline), Visit 4 (Week 2), Visit 5 (Week 4), Visit 6 (Week 8), Visit 7 (Week 12) (End of the Treatment), and Visit 8 (Follow-up 3 weeks). Between the Baseline and Week 12 Visits, patients will attend Visits at Weeks 2, 4 and 8. The randomization will be stratified based on baseline abdominal pain scores (<6 vs≥6 on a 0 to 10 points scale) and IBS subtypes (IBS-D vs IBS-M). The study consists of a Screening Period (1 to 5 weeks), a double-blind Run-in Period (2 weeks), a randomized double-blind Treatment Period (12 weeks), and a Safety Follow-up Period (3 weeks). The follow-up Visit is approximately 21 days after the patient's last dose of Study Treatment (i.e., at Week 15 for patients who complete the Week 12 Visit, or approximately 3 weeks after withdrawal for patients who discontinue the study early). Additionally, Unscheduled Visit(s) may be arranged for patients with study-related safety concerns as needed.

The doses included in this study are 75 mg once daily (QD) or placebo once daily (QD) given to subjects with criteria for diagnosis of IBS-D or IBS-M using the Rome IV criteria, for a treatment period of 12 weeks. Subjects will be randomly allocated at a 1:1 ratio to receive one of two treatments.

The patient Inclusion Criteria are:
1. Capable of giving written informed consent, which includes compliance with the requirements and restrictions listed in the consent form.
2. Female subjects of 18 to 70 years of age, inclusive, at screening.
3. Body weight ≥50 kg (inclusive); body mass index <45.
4. Patient is willing and able to complete the patient diary and quality of life questionnaires.
5. Patient agrees not to participate in another interventional drug or device clinical trial during the study.
6. Female subjects of child bearing potential must agree to use contraception.
7. Patient has completed a colonoscopy according to the American

Gastroenterological Association (AGA) criteria, with no clinically significant findings in the last 5 years.
8. Patient has no clinically significant findings on a physical examination and clinical laboratory tests that could interfere with study participation or confound study assessments, in the opinion of the Investigator. Fecal calprotectin and serum tissue transglutaminase antibody (IgA) must be negative. (Normal complete blood cell count and C-reactive protein is required by Rome IV).
9. Diagnosis of IBS-D or IBS-M according to the Rome IV criteria:
   Recurrent abdominal pain, on average at least 1 day per week in the last 3 months, associated with 2 or more of the following criteria:
      Related to defecation
      Associated with a change in frequency of stool
      Associated with a change in form (appearance) of stool
   Diagnostic criteria must be fulfilled for the last 3 months, with symptom onset at least 6 months before diagnosis.
   Subtyping performed by the predominant stool pattern present in a subject:
      IBS-D: loose, mushy, or watery stools (Bristol Type 6 or 7) for >25% of bowel movements and hard or lumpy stools (Bristol Type 1 or 2) for <25% of bowel movements;
      IBS-M: hard or lumpy stools (Bristol Type 1 or 2) for >25% of bowel movements and loose, mushy, or watery stools (Bristol Type 6 or 7) for >25% of bowel movements;
   The Rome IV diagnostic criteria must be met within the most recent 3 months, with symptom onset at least 6 months before diagnosis.
10. If receiving eluxadoline for the treatment of IBS-D or IBS-M, the dose must have been stable for the past 3 months and continue at the same dose and schedule for the duration of the study.
11. Patient must be willing and able to maintain regular diet for the duration of the study.
12. At the Visit 2 (Baseline), in addition to continuing to meet all the inclusion criteria above at Visit 1 (Screening),
   IBS-D patient must have both additional qualifications based on the 7-day diary: a) weekly average of worst abdominal pain in past 24 hours score of ≥3.0 on a 0 to 10 point scale; and (b) having at least 2 days per week with at least one stool that has a consistency of Type 6 or Type 7 by the Bristol Stool Form Scale.
   IBS-M subject must have a weekly average of “worst abdominal pain in the past 24 hours” score of ≥3.0 on a 0 to 10 point numeric rating scale (NRS).

Certain patient Exclusion Criteria will be used to exclude patients:
1. IBS-C or IBS-U per Rome IV criteria.
2. Chronic idiopathic constipation or functional constipation.
3. Current or history (in the past year) of substance or alcohol abuse, alcoholism, alcohol addiction, or drinks >3 alcoholic beverages per day (one drink defined as 12 ounces of beer, 5 ounces of wine, or 1.5 ounces of distilled spirits).
4. History of clinically relevant liver disease or severe hepatic impairment (Child-Pugh Class C).
5. Pre-existing condition that has altered normal gastrointestinal anatomy (e.g., prior bariatric surgery or gastric banding).
6. History of or suspected mechanical gastrointestinal obstruction or current symptoms suggestive of gastrointestinal obstruction or infection.
7. Diverticulitis within prior 3 months.
8. Structural abnormality of the gastrointestinal tract or a disease (e.g., known small intestine bacterial overgrowth) or condition that can affect gastrointestinal motility.
9. History of a gastrointestinal motility disorder other than IBS (e.g., gastroparesis, intestinal pseudo-obstruction, achalasia, Parkinson's disease, multiple sclerosis, spinal cord injury).
10. Severe constipation or sequelae from constipation.
11. Active duodenal or gastric ulcer.
12. History of solitary rectal ulcer syndrome.
13. Prior history of a gastrointestinal malignancy, inflammatory bowel disease, celiac disease.
14. History of colitis (ischemic, lymphocytic, collagenous or radiation-induced) or hepatic and/or renal function that could interfere with the absorption, metabolism and/or excretion of the study drug (e.g., carcinoid syndrome, amyloidosis).
15. Appendectomy within the past 6 months.
16. Planned gastrointestinal or abdominal surgery within the next 6 months.
17. Co-existing gastroesophageal reflux disease or functional dyspepsia with symptoms predominant to IBS symptoms.
18. History of cholecystectomy and currently receiving eluxadoline.
19. Symptoms or diagnosis of a medical condition other than IBS that may contribute to abdominal pain (e.g., interstitial cystitis; fibromyalgia currently being treated with pregabalin, gabapentin, or neurontin; and endometriosis with uncontrolled abdominal pain).
20. History of pancreatitis, structural diseases of the pancreas (including known or suspected pancreatic duct obstruction), cholecystitis, symptomatic cholelithiasis, known or suspected biliary duct obstruction, or sphincter of Oddi disease or dysfunction.
21. Lactose intolerance not controlled by lactose-free diet.
22. History or evidence of symptomatic arrhythmia, angina/ischemia, coronary artery bypass grafting surgery, percutaneous transluminal coronary angioplasty, cerebrovascular accident, transient ischemic attack, or any clinically significant cardiac disease.
23. Uncontrolled hypertension (sitting systolic blood pressure ≥180 mmHg and/or sitting diastolic blood pressure ≥100 mmHg) or resting heart rate (by pulse) >100 beats per minute.
24. Systolic blood pressure ≥160 mmHg but <180 mmHg is excluded unless deemed by the Investigator as safe to proceed in this study and agreed to by the Sponsor's designated Medical Monitor; must be on stable hypertension medication for at least 90 days.
25. Concurrent malignancy or history of any malignancy (within the last 5 years), except non-metastatic basal or squamous cell carcinoma of the skin that has been treated successfully.
26. Uncontrolled diabetes mellitus defined by a hemoglobin A1C level >8%.
27. Significant psychiatric or psychologic disorder that would preclude meaningful participation in the study (e.g., bipolar disorders or schizophrenia; treated depression is allowed).
28. Known or suspected HIV or AIDS or unexplained alarm symptoms (e.g., anemia, gastrointestinal bleeding, unintentional weight loss, suspected malignancy).
29. Use of any prohibited medications (e.g., subject is using opiates or cannabis products to control their pain, or for any other condition; suitable washout periods from these medications are also described therein); urine drug screen must be negative.
30. Dose change for any medications requiring a stable dose prior to the Screening Visit or plans to initiate or change the dosing of any of these medications during the study.
31. Currently participating or has participated in a study with an investigational compound or device or procedure within 28 days prior to screening.
32. Currently participating in or has participated in a study with vibegron.
33. ALT or AST >2.0 times upper limit of normal (ULN), or bilirubin (total bilirubin) >1.5×ULN (or >2.0×ULN if secondary to Gilbert syndrome or pattern consistent with Gilbert syndrome).
34. Lipase >2×ULN.
35. Estimated glomerular filtration rate <30 mL/min/1.73 m².
36. Women who are pregnant, nursing, or planning a pregnancy during the study.
37. History of sensitivity to any of the study drugs, or components thereof, or a history of drug or other allergy that, in the opinion of the Investigator, contraindicates study participation.
38. At Baseline (Visit 3), noncompliant with dosing during the 2-week Run-in Period (taking <80% or >120% of study medication).
39. Clinically significant medical or surgical history or any condition that could interfere with study participation or confound the assessments in the opinion of the study Investigator.

Subjects will complete two diaries daily, a Bowel Movement Diary and a Pain

Diary. The Bowel Movement Diary will be event-driven (i.e., subjects will record events as they occur) and will capture bowel movement frequency and form, bowel urgency, and recurrent bowel movements. The Pain Diary will prompt subjects each evening to answer a question regarding worst abdominal pain for the past 24 hours using the 0 to 10 point NRS to rate pain and to capture use of rescue medications.

The criteria for evaluating the patients include several endpoints, including primary, secondary, and other efficacy endpoints. The primary efficacy endpoint measures the proportion of Abdominal Pain Intensity (API) Weekly Responders at Week 12. The API weekly responder is defined as a patient who experiences a decrease in weekly average of “worst abdominal pain in the past 24 hours” scores of at least 30% compared with baseline weekly average.

The secondary efficacy endpoints measure the Mean Patient Global Impression Scale (GIS) score at Week 12 and the Proportion of IBS-Quality of Life (QoL) responders at Week 12. The IBS-QoL responder is defined as a patient who has achieved at least a 14-point improvement in IBS-QoL total score. The IBS-QoL consists of 34 items, each with a five-point response scale.

Other efficacy endpoints will include a measure of the following:
Change from baseline to Weeks 2, 4, 8, and 12 in the weekly average of the worst daily API scores
Proportion of API Weekly Responders at Week 2, Week 4 and Week 8.
Change from baseline to Weeks 2, 4, 8, and 12 in the weekly average number of days with pain within 1 hour of eating; defined as the response (yes or no) to the question: Did you experience abdominal pain within 1 hour of eating?
Change from baseline to Weeks 2, 4, 8, and 12 in the weekly average number of days with pain associated with a bowel movement; defined as the response (yes or no) to the question: Did you experience abdominal pain associated with a bowel movement?
Change from baseline in Work Productivity and Activity Impairment (WPAI) score at Weeks 8 and 12.
Mean Patient Global Impression Scale score at Weeks 2, 4 and 8.
Proportion of IBS-QoL responders at Weeks 8 and 12 (responder subject has ≥14-p improvement in IBS-QoL total score).
Change from baseline to Week 12 in the daily average urgency episodes; urgency is defined as the feeling that you need to rush to the bathroom immediately to avoid soiling your pants with a bowel movement.
Change from baseline in daily average of urgency episodes at Weeks 2, 4, 8, and 12.
Change from baseline to Weeks 2, 4, 8, and 12 in the daily average number of bowel movements.
Change from baseline to Weeks 2, 4, 8, and 12 in the number of days with diarrhea (Bristol Type 6 or 7) per week.
For subjects with IBS-D, the proportion of Stool Consistency Weekly Responders at Weeks 2, 4, 8, and 12.
   Stool Consistency Weekly Responder defined as a decrease of at least 50% in the number of days per week with at least 1 stool with consistency of diarrhea (Bristol Type 6 or 7) compared with Baseline.
For subjects with IBS-D, the change from baseline to Weeks 2, 4, 8, and 12 in the weekly number of days with at least 1 stool that has a consistency of diarrhea (Bristol Type 6 or 7).
For subjects with IBS-D, the change from baseline to Weeks 2, 4, 8, and 12 in the weekly number of diarrhea stools (Bristol Type 6 or 7).
For subjects with IBS-M, the change from baseline to Weeks 2, 4, 8, and 12 in the weekly number of days with at least 1 stool with Bristol Type 3, 4, or 5 consistency.
For subjects with IBS-M, the change from baseline to Weeks 2, 4, 8, and 12 in weekly number of stools with Bristol Type 3, 4, or 5 consistency.
Proportion of subjects using rescue medication

The safety endpoints include safety and tolerability parameters such as adverse events (AEs), change in concurrent medications, clinical laboratory values, and vital sign assessments, including blood pressure. An AE is any untoward medical occurrence in a clinical study subject, temporally associated with the use of study drug, whether or not considered related to the study drug. An AE can therefore be any unfavorable and unintended sign (including an abnormal laboratory finding), symptom, or disease (new or exacerbated) temporally associated with the use of study drug.

### Example 7

### A Phase 1, Randomized, Double-Blind, Placebo-Controlled, Two-Period, Crossover Study to Evaluate the Efficacy and Safety of Vibegron Administered Orally for 6 Weeks to Subjects with Irritable Bowel Syndrome (IBS)

The primary objective is to compare the effect of vibegron vs. placebo in subjects with abdominal pain due to irritable bowel syndrome (IBS) on the abdominal pain intensity (API) weekly responder rate at Week 6.

Secondary objectives are the following:
Compare the effect of vibegron vs. placebo in subjects with abdominal pain due to IBS on patient-reported outcomes
Compare the effect of vibegron vs. placebo in subjects with abdominal pain due to IBS on safety endpoints

The primary endpoint is the proportion of API weekly responders at Week 6. An

API Weekly Responder is defined as a subject who experiences a reduction in the weekly average of “worst abdominal pain in the past 24 hours” scores of at least 30% compared with the baseline weekly average. Secondary endpoints are Global Improvement Scale score at Week 6, adverse events (AEs), clinical laboratory values, and vital signs.

### Overall Study Design

This study is a Phase 1, randomized, double-blind, placebo-controlled, two-period, crossover study to evaluate the efficacy and safety of vibegron in adult subjects with IBS. The crossover study is composed of two 6-week treatment periods (Treatment Period 1 and Treatment Period 2) that are separated by a 4-week washout period. A total of approximately 20 subjects will be randomly assigned to one of two treatment sequences (Sequence A [n=10] or Sequence B [n=10]) in a 1:1 ratio at Visit 2 (Day 1). Randomization will be stratified by baseline abdominal pain intensity score (<6 vs ≥6 on a 0- to 10-point numeric rating scale) and IBS subtype.

Subjects in Sequence A (vibegron 75 mg→placebo) first receive vibegron 75 mg once daily (QD) for 6 weeks during Treatment Period 1. After Treatment Period 1, subjects undergo a 4-week washout period in which subjects do not receive any study medication. Following the washout period, Treatment Period 2 commences in which subjects receive placebo QD for 6 weeks.

Subjects in Sequence B (placebo→vibegron 75 mg) first receive placebo QD for 6 weeks during Treatment Period 1. After Treatment Period 1, subjects undergo a 4-week washout period in which subjects do not receive any study medication. Following the washout period, Treatment Period 2 commences in which subjects receive vibegron 75 mg QD for 6 weeks.

The study consists of screening, Treatment Period 1 (6 weeks), washout (4 weeks), and Treatment Period 2 (6 weeks).

### Number of Subjects

In total, approximately 20 subjects are randomized in a 1:1 ratio to either Treatment Sequence A or B (10 per sequence). Assuming a total of 10% of subjects discontinue prior to the end of Treatment Period 2 (for any reason), there are approximately 18 evaluable IBS subjects at the end of the study.

### Study Drug Groups and Study Duration

Double-blind Treatment: 1 vibegron (75 mg) and matched placebo tablet QD for up to 6 weeks each in both Treatment Sequence A (vibegron 75 mg→placebo) and Treatment Sequence B (placebo→vibegron 75 mg).

### 1. Objectives & Endpoints

| Objectives | Endpoints |
|---|---|
| Primary | |
| To compare the effect of vibegron | Proportion of IBS subjects who are API weekly |
| vs placebo in subjects with | responders at Week 6 |
| abdominal pain due to IBS on | An API Weekly Responder is defined as a subject |
| the API weekly responder rate | who experiences a decrease in the weekly average |
| at Week 6 | of “worst abdominal pain in the past 24 hours” |
| | scores of at least 30% compared with the baseline |
| | weekly average |
| Secondary | |
| To compare the effect of vibegron | GIS score at Week 6 for IBS subjects |
| vs placebo in subjects with | |
| abdominal pain due to IBS on | |
| patient-reported outcomes | |
| To compare the effect of vibegron | AEs, clinical laboratory values, vital signs |
| vs placebo in subjects with | |
| abdominal pain due to IBS on | |
| safety endpoints | |

AE(s) = adverse event(s);
API = Abdominal Pain Intensity;
GIS = Global Improvement Scale;
IBS = irritable bowel syndrome

| Assessment | Timing | Measurement |
|---|---|---|
| Abdominal Pain | Screening and double-blind | 0- to 10-point numeric rating |
| Intensity (API) | Treatment Period | scale: |
| [Primary] | | 0 = no pain |
| | | 10 = worst possible pain |
| | | Subjects will record worst |
| | | abdominal pain on evening Pain |
| | | Diary in response to daily prompt |
| PRO | Screening and double-blind | GIS [Secondary] |
| Questionnaires | Treatment Period | WPAI |
| | | IBS-QoL |
| Bowel Urgency | Screening and double-blind | Urgency episodes recorded on |
| | Treatment Period | Bowel Movement Diary |
| Recurrent Bowel | Screening and double-blind | Number of days per week with |
| Movements | Treatment Period | recurrent bowel movement, |
| | | defined as >1 bowel movement |
| | | within any 60-minute period; |
| | | bowel movements recorded on |
| | | Bowel Movement Diary |
| Stool Frequency | Screening and double-blind | Bowel movements recorded on |
| | Treatment Period | Bowel Movement Diary |
| Stool | Screening and double-blind | Bristol Stool Form consistency |
| Consistency | Treatment Period | recorded on Bowel Movement |
| | | Diary |

GIS = Global Improvement Scale;
IBS-QoL = IBS quality of life assessment;
PRO = patient-reported outcomes;
WPAI = Work Productivity and Activity Impairment

### 2. Study Population

The study is conducted in males and females with IBS. Specific inclusion and exclusion criteria are specified below.

The patient Inclusion Criteria are:
1. Capable of giving written informed consent, which includes compliance with the requirements and restrictions listed in the informed consent form, including agreeing to patient registry verification.
2. Male or female, 18 to 70 years of age, inclusive
3. Body weight ≥50 kg; body mass index <45
4. Diagnosis of IBS-D, IBS-M, or IBS-C according to the Rome IV criteria:
   Recurrent abdominal pain, on average at least 1 day per week in the last 3 months, associated with 2 or more of the following criteria:
      Related to defecation
      Associated with a change in frequency of stool
      Associated with a change in form (appearance) of stool
   Diagnostic criteria must be fulfilled for the last 3 months, with symptom onset at least 6 months before diagnosis.
   Subtyping performed by the predominant stool pattern present in a subject:
      IBS-D: loose, mushy, or watery stools (Bristol Type 6 or 7) for >25% of bowel movements and hard or lumpy stools (Bristol Type 1 or 2) for <25% of bowel movements
      IBS-M: hard or lumpy stools (Bristol Type 1 or 2) for >25% of bowel movements and loose, mushy, or watery stools (Bristol Type 6 or 7) for >25% of bowel movements
      IBS-C: hard or lumpy stools (Bristol Type 1 or 2) for >25% of bowel movements and loose, mushy, or watery stools (Bristol Type 6 or 7) for <25% of bowel movements
5. Has completed a colonoscopy according to the American Gastroenterological Association criteria, with no clinically significant findings in the last 5 years
6. Willing and able, as assessed by the Investigator, to follow study instructions, including completing the subject diaries and quality of life questionnaires and attending all study visits
7. Willing and able to maintain regular diet for the duration of the study
8. Has no clinically significant findings on a physical examination or clinical laboratory tests that could interfere with study participation or confound study assessments, in the opinion of the Investigator. Fecal calprotectin and serum tissue transglutaminase antibody (IgA) must be negative. (Normal complete blood cell count and C-reactive protein is required by Rome IV)
9. Women of childbearing potential must agree to use a highly effective method(s) of contraception
10. Agrees not to participate in another clinical trial while participating in this study
11. At Screening, as reported in the subject's daily pain diary and daily bowel movement diary:
   IBS-D subject must have both a weekly average of “worst abdominal pain in the past 24 hours” score of ≥3.0 on a 0- to 10-point NRS and must have at least 2 days per week with at least one stool with a consistency of Bristol Type 6 or 7
   IBS-M subject must have a weekly average of “worst abdominal pain in the past 24 hours” score of ≥3.0 on a 0- to 10-point NRS
   IBS-C subject must have both a weekly average of “worst abdominal pain in the past 24 hours” score of ≥3.0 on a 0- to 10-point NRS and must have at least 2 days per week with at least one stool with a consistency of Bristol Type 1 or 2

### 2.1 Exclusion Criteria

Subjects are excluded from the study if any of the following criteria apply at Screening, unless otherwise noted:
1. History of chronic idiopathic constipation or functional constipation other than IBS-C
2. Current or history (in the past year) of substance or alcohol abuse, alcoholism, alcohol addiction, or drinks >3 alcoholic beverages per day (one drink defined as 12 ounces of beer, 5 ounces of wine, or 1.5 ounces of distilled spirits)
3. History of clinically relevant liver disease or severe hepatic impairment (Child-Pugh Class C)
4. Pre-existing condition that has altered normal gastrointestinal anatomy (e.g., prior bariatric surgery or gastric banding, solitary rectal ulcer syndrome)
5. History of or suspected mechanical gastrointestinal obstruction or current symptoms suggestive of gastrointestinal obstruction or infection
6. Diverticulitis within prior 3 months
7. Structural abnormality of the gastrointestinal tract or a disease (e.g., known small intestine bacterial overgrowth) or condition that can affect gastrointestinal motility
8. History of a gastrointestinal motility disorder other than IBS (e.g., gastroparesis, intestinal pseudo-obstruction, achalasia, Parkinson's disease, multiple sclerosis, spinal cord injury)
9. Severe constipation or sequelae from constipation
10. Active duodenal or gastric ulcer
11. History of solitary rectal ulcer syndrome
12. Prior history of a gastrointestinal malignancy, inflammatory bowel disease, celiac disease
13. History of colitis (ischemic, lymphocytic, collagenous or radiation-induced) or hepatic and/or renal function that could interfere with the absorption, metabolism and/or excretion of the study drug (e.g., carcinoid syndrome, amyloidosis)
14. Appendectomy within the past 6 months
15. Planned gastrointestinal or abdominal surgery within the next 6 months
16. Co-existing gastroesophageal reflux disease or functional dyspepsia with symptoms predominant to IBS symptoms
17. Symptoms or diagnosis of a medical condition other than IBS that may contribute to abdominal pain (e.g., interstitial cystitis; fibromyalgia currently being treated with pregabalin or gabapentin; and endometriosis with uncontrolled abdominal pain)
18. History of pancreatitis, structural diseases of the pancreas (including known or suspected pancreatic duct obstruction), cholecystitis, symptomatic cholelithiasis, known or suspected biliary duct obstruction, or sphincter of Oddi disease or dysfunction
19. Lactose intolerance not controlled by lactose-free diet
20. History or evidence of symptomatic arrhythmia, angina/ischemia, coronary artery bypass grafting surgery, percutaneous transluminal coronary angioplasty, cerebrovascular accident, transient ischemic attack, or any clinically significant cardiac disease
21. Clinically significant electrocardiogram (ECG) abnormality that, in the opinion of the Investigator, exposes the subject to risk by participating in the study
22. Uncontrolled hypertension (sitting systolic blood pressure ≥180 mmHg and/or sitting diastolic blood pressure ≥100 mmHg) or resting heart rate (by pulse) >100 beats per minute
23. Systolic blood pressure ≥160 mmHg but <180 mmHg is excluded unless deemed by the Investigator as safe to proceed in this study and agreed to by the Sponsor's designated Medical Monitor; must be on stable hypertension medication for at least 90 days
24. Concurrent malignancy or history of any malignancy (within the last 5 years), except non-metastatic basal or squamous cell carcinoma of the skin that has been treated successfully
25. Uncontrolled diabetes mellitus defined by a hemoglobin A1C level >8%
26. Significant psychiatric or psychologic disorder that would preclude meaningful participation in the study (e.g., bipolar disorders or schizophrenia; treated depression is allowed)
27. Known or suspected HIV or AIDS or unexplained alarm symptoms (e.g., anemia, gastrointestinal bleeding, unintentional weight loss, suspected malignancy)
28. Use of any medications for the treatment of IBS (e.g., eluxadoline, rifaximin, opioids, etc.); suitable washout periods of at least 14 days from these medications are required
29. Currently participating in or has participated in a study with an investigational compound or device or procedure within 28 days prior to screening
30. Currently participating in or has participated in a study with vibegron
31. ALT or AST >2.0 times upper limit of normal (ULN), or bilirubin (total bilirubin) >1.5×ULN (or >2.0×ULN if secondary to Gilbert syndrome or pattern consistent with Gilbert syndrome)
32. Lipase >2×ULN
33. Estimated glomerular filtration rate <30 mL/min/1.73 m²
34. Women who are pregnant, nursing, or planning a pregnancy during the study
35. History of sensitivity to any of the study drugs, or components thereof, or a history of drug or other allergy that, in the opinion of the Investigator, contraindicates study participation
36. Clinically significant medical or surgical history or any condition that could interfere with study participation or confound the assessments in the opinion of the study Investigator

Having now fully described this invention, it will be understood by those of ordinary skill in the art that the same can be performed within a wide and equivalent range of conditions, formulations and other parameters without affecting the scope of the invention or any embodiment thereof.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

All patents, patent applications, and other publications cited herein are fully incorporated by reference herein in their entirety.

What is claimed is:

1. A method of treating or preventing pain associated with irritable bowel syndrome, the method comprising orally administering to a subject in need thereof vibegron.

2. The method of claim 1, wherein the method comprises orally administering an amount of from about 1 mg to about 1000 mg of vibegron.

3. The method of claim 2, wherein the amount of vibegron is about 25 mg to about 150 mg.

4. The method of claim 3, wherein the amount of vibegron is about 50 mg to about 100 mg.

5. The method of claim 4, wherein the amount of vibegron is about 50 mg.

6. The method of claim 4, wherein the amount of vibegron is about 75 mg. The method of claim 4, wherein the amount of vibegron is about 100 mg.

8. The method of any one of claims 1 to 7, wherein the subject has a symptom selected from the group consisting of abnormal stool frequency, abnormal stool form or consistency, abnormal stool passage, abnormal urgency, passage of mucus, bloating, feeling of abdominal distension, abdominal discomfort, bowel movements with incomplete evacuation, and combinations thereof.

9. The method of any one of claims 1 to 8, wherein the subject experiences an improvement in abnormal stool passage following administration of vibegron.

10. The method of any one of claims 1 to 9, wherein the subject experiences a decreased urgency following administration of vibegron.

11. The method of any one of claims 1 to 10, wherein the subject experiences a decreased passage of mucus, bloating, and/or feelings of abdominal distension following administration of vibegron.

12. The method of any one of claims 1 to 11, wherein the subject experiences a decrease in abdominal discomfort following administration of vibegron.

13. The method of any one of claims 1 to 12 wherein the subject experiences a decrease in bowel movements with incomplete evacuation following administration of vibegron.

14. The method of any one of claims 1 to 13, wherein the subject has a symptom of abdominal discomfort, wherein the abdominal discomfort has two features selected from the group consisting of relieved with defecation, onset associated with a change in frequency of stool, and onset associated with a change in form or appearance of stool.

15. The method of any one of claims 1 to 14, wherein the subject experiences a change in stool frequency over a treatment period.

16. The method of any one of claims 1 to 15, wherein the subject experiences an increase in stool frequency over a treatment period.

17. The method of any one of claims 1 to 15, wherein the subject experiences a decrease in stool frequency over a treatment period.

18. The method of any one of claims 1 to 14, wherein the subject does not experience a change in stool frequency over a treatment period.

19. The method of any one of claims 1 to 18, wherein the subject experiences a change in stool consistency over a treatment period.

20. The method of any one of claims 1 to 19, wherein the subject experiences a decrease in score on the Bristol Stool Form Scale over a treatment period.

21. The method of any one of claims 1 to 19, wherein the subject experiences an increase in score on the Bristol Stool Form Scale over a treatment period.

22. The method of any one of claims 1 to 18, wherein the subject does not experience a change in stool consistency over a treatment period.

23. The method of any one of claims 1 to 22, wherein the subject experiences a change in gastrointestinal and/or colonic transit over a treatment period.

24. The method of any one of claims 1 to 23, wherein the subject experiences an increase in gastrointestinal and/or colonic transit over a treatment period.

25. The method of any one of claims 1 to 23, wherein the subject experiences a decrease in gastrointestinal and/or colonic transit over a treatment period.

26. The method of any one of claims 1 to 25, wherein the subject experiences a change in somatostatin plasma concentration over a treatment period.

27. The method of any one of claims 1 to 26, wherein the subject does not experience an increase in an adverse event selected from the group consisting of nasopharyngitis, headache, nausea, gastroenteritis, diarrhea, constipation, and a combination thereof.

28. The method of any one of claims 10 to 27, wherein the treatment period is selected from the group consisting of 2, 4, 6, 8, 10, 12, 14, 16, 18, and 20 weeks.

29. The method of any one of claims 1 to 28, wherein the subject experiences a decrease in the weekly average abdominal pain score compared with baseline.

30. The method of claim 29, wherein the subject experiences a decrease of at least about 30 percent, a decrease of at least about 40 percent, a decrease of at least about 50 percent, a decrease of at least about 60 percent, a decrease of at least about 70 percent, a decrease of at least about 80 percent, a decrease of at least about 90 percent, or a decrease of about100 percent in the weekly average abdominal pain score compared with baseline.

31. The method of any one of claims 1 to 30, wherein the subject is a human.

32. The method of claim 31, wherein the human is a female.

33. The method of claim 31, wherein the human is a male.

34. The method of any one of claims 1 to 33, wherein the subject is under the age of about 18 years.

35. The method of claim 34, wherein the subject is between the age of about 6 years to about 18 years.

36. The method of any one of claims 1 to 33, wherein the subject is over the age of about 18 years.

37. The method of claim 36, wherein the subject is between the age of about 18 years to about 50 years.

38. The method of any one of claims 1 to 37, wherein the subject suffers from IBS-D or IBS-M.

39. The method of claim 38, wherein the subject suffers from IBS-D.

40. The method of claim 38, wherein the subject suffers from IBS-M.

41. The method of any one of claims 1 to 37, wherein the subject suffers from IBS-C.

42. The method of any one of claims 1 to 41, wherein the subject suffers from severe renal impairment.

43. The method of any one of claims 1 to 42, wherein the subject suffers from moderate renal impairment.

44. The method of any one of claims 1 to 43, where the subject is concomitantly receiving, taking or otherwise being exposed to at least one additional therapeutic agent.

45. The method of claim 44, wherein the at least one therapeutic agent is selected from a therapeutic agent for irritable bowel syndrome, a therapeutic agent for diarrhea, a therapeutic agent for constipation, an ameliorating agent for abdominal pain, a digestive tract motility regulator, a digestive tract motility activator, an anti-depressant agent, an anti-anxiety agent, or combinations thereof.

46. The method of claim 44, wherein the at least one therapeutic agent is selected from the group consisting of a guanylate cyclase 2C agonist, a μ-opioid receptor agonist, κ-opioid receptor agonist, and combinations thereof.

47. The method of claim 44, wherein the at least one therapeutic agent is a CYP3A/P-glycoprotein inhibitor.

48. The method of any one of claims 1 to 47, wherein vibegron is administered once per day.

49. The method of any one of claims 1 to 47, wherein vibegron is administered twice per day.

50. The method of any one of claims 1 to 49, wherein vibegron is administered with a meal.

51. The method of any one of claims 1 to 49, wherein vibegron is administered without a meal.

52. The method of any one of claims 1 to 51, wherein vibegron is administered as a free base.

53. The method of any one of claims 1 to 51, wherein vibegron is administered as a pharmaceutically acceptable salt thereof.

54. Vibegron for use in the treatment or prevention of a pain associated with irritable bowel syndrome.

55. Vibegron for use in the treatment or prevention of a pain associated with irritable bowel syndrome as claimed in claim 54 wherein vibegron is administered in an amount of from about 1 mg to about 1000 mg.

56. Vibegron for use in the treatment or prevention of a pain associated with irritable bowel syndrome as claimed in claim 54 wherein the pain associated with irritable bowel syndrome is caused by a symptom selected from the group consisting of abnormal stool frequency, abnormal stool form or consistency, abnormal stool passage, abnormal urgency, passage of mucus, bloating, feeling of abdominal distension, abdominal discomfort, bowel movements with incomplete evacuation, and combinations thereof.

57. Vibegron for use in the treatment or prevention of a pain associated with irritable bowel syndrome as claimed in claim 54 wherein the pain associated with irritable bowel syndrome is associated with abdominal discomfort, wherein the abdominal discomfort has two features selected from the group consisting of relieved with defecation, onset associated with a change in frequency of stool, and onset associated with a change in form or appearance of stool.

58. Vibegron for use in the treatment or prevention of a pain associated with irritable bowel syndrome as claimed in claim 54 wherein the pain is associated with IBS-D or IBS-M or IBS-C.

59. Vibegron for use in the treatment or prevention of a pain associated with irritable bowel syndrome as claimed in claim 54 wherein the vibegron is provided as a combined medicament with an additional therapeutic agent for use in the treatment of irritable bowel syndrome, a therapeutic agent for diarrhea, a therapeutic agent for constipation, an ameliorating agent for abdominal pain, a digestive tract motility regulator, a digestive tract motility activator, an anti-depressant agent, or an anti-anxiety agent wherein the vibegron and the therapeutic agent are provided simultaneously, sequentially or separately.

60. Vibegron for use in the preparation of a medicament for the treatment or prevention of a pain associated with irritable bowel syndrome.
